(19) United States
(12) Patent Application Publication
(10) Pub. No.: US 2024/0159750 A1
(43) Pub. Date: May 16, 2024

(54) METHODS FOR DIAGNOSIS AND TREATMENT OF COVID-19

(71) Applicant: MARSHALL UNIVERSITY RESEARCH CORPORATION, Huntington, WV (US)

(72) Inventors: Wei LI, Huntington, WV (US); Hong YUE, Huntington, WV (US)

(21) Appl. No.: 18/273,158

(22) PCT Filed: Jan. 19, 2022
(86) PCT No.: PCT/US2022/012878

§ 371 Date: Jul. 19, 2023

Related U.S. Application Data
(60) Provisional application No. 63/139,021, filed on Jan. 19, 2021.

(51) Int. Cl. C12Q 1/48

(52) U.S. Cl. CPC C12Q 1/48; G01N 2333/165; G01N 2333/91142

(57) ABSTRACT

Methods for diagnosis and treatment of COVID-19 acuity in a subject include the identification of a subject as having Nan increased expression level and/or activity of thymidine phosphorylase in a biological sample obtained from the subject. An effective amount of a therapeutic agent that reduces the expression level or activity of thymidine phosphorylase can the be administered to the subject.

### RELATED APPLICATIONS

This application claims priority from U.S. Provisional Application Ser. No. 63/139,021, filed Jan. 19, 2021, the entire disclosure of which is incorporated herein by this reference.

### GOVERNMENT INTEREST

This invention was made with government support under grant number R15HL145573, awarded by the National Institutes of Health, and grant number U54GM104942, awarded by the National Institute of General Medical Sciences. The government has certain rights in the invention.

### TECHNICAL FIELD

The presently-disclosed subject matter generally relates to methods for diagnosis and treatment of COVID-19. In particular, certain embodiments of the presently-disclosed subject matter relate to methods for diagnosis and treatment of COVID-19 whereby COVID-19 acuity is assessed and/or treated based on a determination of thymidine phosphorylase (TYMP) expression level or activity.

### BACKGROUND

COVID-19 is a viral respiratory illness caused by the new Severe Acute Respiratory Syndrome Coronavirus 2 (SARS-CoV-2). By Oct. 1, 2021, there were more than 230 million confirmed infections globally and more than 4.7 million deaths. This pandemic is more severe in the Western world, especially in the U.S., which faced a fourth surge beginning from early July 2021, with approximately 44 million confirmed cases and more than 0.7 million COVID-19-associated deaths. Although vaccines are being distributed globally, due to the fast mutation of the virus, the current vaccination schedule cannot lead to long-term herd immunity to stop the pandemic and repeated vaccinations or reboots have become necessary. Vaccination has also been dramatically delayed in developing countries, thus exacerbating the issue.

Additionally, the fundamental mechanisms behind COVID-19 pathogenicity are still poorly understood. Large-scale reports have characterized the symptoms, comorbidities, and clinical outcomes of this illness and suggested that patients with cardiovascular- or diabetes-associated comorbidities are at a higher risk for developing advanced COVID-19 with admission to intensive care. The three most prevalent comorbid conditions are hypertension, obesity, and diabetes. Currently, however, no effective COVID-19 treatment regimens have been established, and an interim report for the WHO (World Health Organization) solidarity trial has indicated that remdesivir, hydroxychloroquine, lopinavir, and interferon regimens have little or no effect on the hospitalized COVID-19 patients. Patients with advanced COVID-19 often die from acute respiratory distress syndrome (ARDS) or multi-organ failure. The causes of such ARDS and multi-organ failure are not entirely clear; however, emerging evidence indicates that an inflammatory “cytokine storm” and diffuse microthrombi formation in the small arteries of the lungs or pulmonary embolism play important roles in the COVID-19 milieu. Viral infections evoke a systemic inflammatory response that may cause an imbalance between pro-coagulant and anti-coagulant homeostatic mechanisms. Indeed, a cohort study reported that 71% of non-surviving COVID-19 patients had disseminated intravascular coagulation based on the criteria of the International Society on Thrombosis and Haemostasis (ISTH). Although only 0.6% of the survivors had disseminated intravascular coagulation, accumulating evidence further suggested that SARS-CoV-2 positive individuals have an increased risk of heart attack and stroke, especially in younger patients.

While vaccines are still currently being distributed, accumulating data has also recently confirmed the anecdotal findings that vaccine-associated clot formation happens, suggesting that the SARS-CoV-2 spike protein, an antigen used for generating neutralizing antibodies or expressed via vaccination, may play a mechanistic role in enhancing thrombosis in the COVID-19 milieu. This will be a further disadvantage if mutated SARS-CoV-2 strains continue to adapt in the human population for years or decades and repeated vaccinations and reboots continue to be necessary. Therefore, novel mechanism-based diagnostic and therapeutic strategies for COVID-19 are urgently needed, and would be both highly desirable and beneficial.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of such features.

The presently-disclosed subject matter includes methods for diagnosis and treatment of COVID-19 whereby COVID-19 acuity is assessed and/or treated based on a determination of thymidine phosphorylase (TYMP) expression level or activity. In some embodiments, a method for diagnosis or prognosis of COVID-19 acuity is provided. In some embodiments, a method for diagnosis or prognosis of COVID-19 acuity in a subject having COVID-19 is provided that includes the steps of providing a biological sample from the subject, and then determining an expression level or activity of thymidine phosphorylase in the sample. Subsequent to comparing the expression level or activity of thymidine phosphorylase in the sample, if present, to a control expression level or activity of thymidine phosphorylase, the subject is then diagnosed as having or at an increased risk of COVID-19 acuity if there is a measurable difference in the expression level or activity of the thymidine phosphorylase in the sample as compared to the control level. In some embodiments, the COVID-19 acuity is characterized by thrombosis, inflammation, and/or organ damage in the subject and, in certain embodiments, the biological sample obtained from the subject (e.g., a human subject) comprises blood, plasma, or serum.

To determine the expression level or activity in the sample of the thymidine phosphorylase, in some embodiments, determining the expression level or activity comprises determining the expression level or activity in the sample of the thymidine phosphorylase using mass spectrometry (MS) analysis, immunoassay analysis, or both. In some embodiments, a treatment for the COVID-19 is selected or modified based on the determined expression level or activity of the thymidine phosphorylase.

In some further embodiments of the diagnostic methods described herein, the methods comprise the step of determining an expression level or activity of C-reactive protein (CRP) and/or lactate dehydrogenase (LDH) in the biological sample from the subject. In some embodiments, the methods further comprise a step of administering to the subject a therapeutic agent capable of affecting the expression level or activity of thymidine phosphorylase in the subject. In some embodiments, that therapeutic agent is tipiracil.

Further provided, in some embodiments, are methods for determining whether to initiate or continue prophylaxis or treatment of COVID-19 in a subject whereby a series of biological samples is provided over a time period from the subject. The series of biological samples is then analyzed to determine an expression level or activity in each of the biological samples of thymidine phosphorylase, and any measurable change in the expression level or activity of thymidine phosphorylase in each of the biological samples is compared to thereby determine whether to initiate or continue the prophylaxis or therapy of the COVID-19. In some embodiments, an expression level or activity of C-reactive protein (CRP) and/or lactate dehydrogenase (LDH) is also determined in the sample, and whether to initiate or continue prophylaxis or therapy of the COVID-19 can be determined based on the expression level or activity of CRP or LDH in the sample.

Still further provided, in some embodiments of the presently-disclosed subject matter, are methods for treating COVID-19 in a subject. In some embodiments, a method for treating COVID-19 in a subject is provided that includes the steps of identifying a subject as having an increased expression level and/or activity of thymidine phosphorylase in a biological sample obtained from the subject, and administering an effective amount of a therapeutic agent that reduces the expression level or activity of thymidine phosphorylase in the subject. In some embodiments of the therapeutic methods, reducing the expression level or activity of thymidine phosphorylase comprises administering to the subject an effective amount of a thymidine phosphorylase inhibitor such as, in certain embodiments, tipiracil. In some embodiments, administering the effective amount of the therapeutic agent reduces an amount of inflammation, D-dimer formation, organ damage, and/or thrombosis in the subject.

Further included in the presently-disclosed subject matter are assays for assessing COVID-19 acuity in a subject as well as methods for screening for a compound useful for treating COVID-19. In some embodiments, an assay for assessing COVID-19 acuity in a subject is provided that comprises the steps of applying an agent capable of affecting detection of an expression level or activity of thymidine phosphorylase in a biological sample obtained from a subject, and determining the expression level or activity of thymidine phosphorylase in the biological sample. In other embodiments, a method for screening for a compound useful for treating COVID-19 is provided that comprises the steps of contacting a cell with an effective amount of a test compound, and detecting an expression level or activity level of thymidine phosphorylase in the cell in the presence of the test compound.

Further features and advantages of the present invention will become evident to those of ordinary skill in the art after a study of the description, figures, and non-limiting examples in this document.

### BRIEF DESCRIPTION OF THE DRAWINGS

FIG. 1 is a schematic diagram showing TYMP expression induced by the SARS-CoV-2 spike protein plays an important mechanistic role in the development of severe COVID-19 (TYMP, thymidine phosphorylase; SP-R, spike protein receptor; AEC-T2, type II alveolar epithelial cells) and where the darker arrows show an enhancing effect and the where the lighter arrows show a modulating effect.

FIGS. 2A-2C include graphs showing that TYMP expression is acuity-dependently increased in COVID-19 patients in the early phase and is a valuable marker for diagnosing severe COVID-19, where data was extracted from the MGH Emergency Department COVID-19 Cohort with Olink Proteomics. Patients were stratified based on days after hospitalization (FIG. 2A) or acuity at day 0 assessed based on the WHO Score (FIG. 2B). A1=death within 28 days; A2=intubated, ventilated, survived to 28 days; A3=non-invasive ventilation or high-flow nasal cannula; A4=hospitalized, supplementary 02 required; A5=hospitalized, no supplementary 02 required; A6=not hospitalized. FIG. 2C provides a ROC analysis based on TYMP expression on Day 0.

FIGS. 3A-3B are graphs showing plasma TYMP levels are correlated with thrombotic events and the presence of respiratory symptoms in patients with COVID-19. TYMP data were sorted based on plasma levels of D-dimer (FIG. 3A) or the presence or absence of respiratory symptoms (FIG. 3B). TYMP expression was analyzed based on the defined groups.

FIGS. 4A-4B are graphs showing plasma TYMP levels are correlated with inflammatory and tissue damage markers in patients with COVID-19. TYMP expression was stratified and analyzed based on plasma levels of CRP (C-reactive protein) (FIG. 3A) or LDH (lactate dehydrogenase) (FIG. 3B).

FIGS. 5A-5C are graphs showing spike protein (SP) and its receptor binding domain (RBD) upregulates TYMP expression in BEAS-2B, human bronchial epithelial cells in vitro and that inhibition of TYMP dramatically attenuated SP-enhanced thrombosis in vivo. COS-7 cells were transiently transfected with plasmid pCDNA3.1 encoding either the RBD domain or the full-length SARS-CoV-2 SP, or empty vector only as control, and cells were lysed in PBS (without any proteinase or phosphatase inhibitor) 24 h later and used for the following studies. FIG. 5A shows the results for BEAS-2B cells in 6 cm plates that were treated with 100 μg of total cell lysates prepared above for 24 hours and where TYMP expression was examined by western blot. Blot represents two independent experiments. FIG. 5B shows the results for BEAS-2B cells in 6-well plates that were treated with 10, 50, and 100 μg RBD containing COS-7 cell lysates or control cell lysate for 24 hours, and where TYMP expression was examined. FIG. 5C shows the results for ACE2^{TG} mice that were treated with a single dose of control (pCDNA3.1 transfected) or SP-transfected COS-7 cell lysate, 500 μg/mouse, through intraperitoneal injection. Some SP-treated mice were also gavage fed tipiracil (TPI) in a dose of 1 mg/kg/day. The mice were then subjected to the FeCl₃ injury-induced carotid artery thrombosis model 3 days later.

FIGS. 6A-6D include a schematic diagram and images showing SARS-CoV-2 infection-induced microthrombi in alveolar capillaries, where FIG. 6A is a schematic diagram showing the alveolocapillary system and where FIGS. 6B-6D show TNFα-induced immunothrombi formation at the juncture of two capillaries in the cremaster muscle. The thrombi grew bigger and disassociated from the original site, forming an embolus. Arrows show thrombi and blood flow direction. Platelets were labeled with rhodamine 6G (red), and leukocytes were labeled with green cell tracker.

FIGS. 7A-7D include images showing TYMP is expressed by AEC-T2 and bronchia epithelium and increased in the inflammatory asthmatic lung. FIG. 7A shows TYMP immunohistochemically stained in normal human lung sections. Data were adopted from “The Human Protein Atlas” (CAB002518). Arrows indicate AEC-T2 cells. Insert shows bronchial epithelium. Brown indicates TYMP positive staining. FIG. 7B shows human normal lung sections fluorescently double-stained for TYMP and pro-surfactant protein C (PSP-C). Nuclei were stained with DAPI. FIGS. 7C-7D show lung sections prepared from normal or ovalbumin-induced asthmatic mice and that were used for immunohistochemical staining of TYMP (Brown).

FIG. 8 includes images and a diagram showing SARS-CoV-2 SP and RBD lead to ACE2 reduced sizes. COS-7 cells were transfected with the plasmids as indicated, and ACE2 expression was determined by western blotting with an anti-myc antibody. Actin was used as loading control. Total 6-ug plasmid was used for each 10-cm dish. In the combination of two plasmids, 3-μg for each was used. The blot represents three repeats. Arrows indicate C9-tagged SP. Some samples were loaded in mirror (same color) for confirming band sizes accurately.

FIGS. 9A-9D includes images and a diagram showing SARS-CoV-2 SP leads to formation of a positive feedback loop between TYMP and NF-κB. In FIGS. 9A-9B, C2 and PC cells were synchronized by serum-starvation for 24 hours and then stimulated with TLR1/2 (FIG. 9A) or TLR4 (FIG. 9B) signaling agonists for the indicated times. In FIG. 9C, BEAS-2B cells were treated with cell lysate prepared from COS-7 cells transfected with pCDNA3.1-SP-S1RBD-sfGFP. In FIG. 9D, a positive loop presents between TYMP and NF-kB in host cells primed with SP.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

The present application can “comprise” (open ended), “consist of” (closed ended), or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently-disclosed subject matter is based, at least in part, on an analysis of preclinical and clinical studies showing that thymidine phosphorylase (TYMP) plays an important role in the SARS-CoV-2-induced inflammation and the COVID-19 associated thrombosis. COVID-19 is a viral respiratory illness caused by the new Severe Acute Respiratory Syndrome coronavirus 2 (SARS-CoV-2). Reduced platelet counts and elevated D-dimer have indicated that excessive activation of the coagulation system or platelet-rich thrombosis occurs with COVID-19. COVID-19 patients have high plasma fibrinogen levels, elevated antiphospholipid antibodies, capillary endothelial cell dysfunction, and elevated serum neutrophil extracellular traps, all risk factors for thrombosis. Microthrombi form in the lungs, lower limbs, hands, brain, liver, and kidneys of COVID-19 patients, suggesting a high rate of arterial thrombosis. Thrombi formed in the lung microvascular venous system can leave their original site to form arterial thromboembolisms. Consequently, the administration of anti-platelet and anti-coagulant medications to critically-ill COVID-19 patients has produced some clinical benefits. Accumulating data from independent laboratories have demonstrated that platelets are super-reactive in COVID-19 patients; however, the mechanism is unknown.

SARS-CoV-2 uses the ACE2 receptor and TMPRSS2 (transmembrane protease, serine 2), a serine protease for spike protein (“SP”) priming, to enter host cells. The binding of SARS-CoV-2 to ACE2 is mediated by the coronavirus SP, a class I fusion protein. The SP contains an N-terminal S1 surface subunit, which harbors the receptor binding domain (“RBD”), and an S2 C-terminal transmembrane subunit, which contains the functional elements required for host-viral membrane fusion, allowing viral genomes to enter host cells. The S2 subunit is the most conserved region of the protein, whereas the S1 subunit diverges in sequence even among species of a single coronavirus. The S1 subunit contains two subdomains: an N-terminal domain and a C-terminal domain. Both can function as RBDs and bind various proteins and sugars. Because binding of the host cell and fusion to their membrane is an important step in viral infections, the SP has been used as an antigen for generating neutralizing antibodies and vaccines. However, due to the fast mutation rates, the vaccines and neutralizing antibodies may lose their protective effect against newly mutant strains. In addition, accumulating data confirmed the anecdotal findings that vaccine-associated clot formation happens in all four major COVID-19 vaccines and have indicated that that the SP may play a role in vaccine-associated thrombosis. Interestingly, it is appreciated that ACE2 activation has antithrombotic activity, and ACE2 activation reduces platelet adhesion to the vessel wall and attenuates thrombus formation in mice.

Both angiogenesis and thrombosis happen in the lungs of COVID-19 patients. TYMP is significantly increased in the lung tissues and is adjacent to the location of thrombosis and angiogenesis, indicating that locally increased TYMP may contribute to both the thrombotic and angiogenic properties in the COVID-19 milieu. Thymidine phosphorylase (TYMP), also known as platelet-derived endothelial cell growth factor, is a cytoplasmic protein highly expressed in platelets. TYMP is expressed by macrophages, stromal and endothelial cells, and some epithelial cells, but not red blood cells. TYMP is an enzyme in the pyrimidine salvage pathway and reversibly catalyzes the conversion of thymidine to thymine. TYMP lacks an amino-terminal hydrophobic leader sequence required for cell secretion and is mainly found inside the cell. TYMP has pro-angiogenic effects, and it is appreciated that TYMP has a signaling function in platelets, and systemic TYMP deficiency or inhibition significantly reduces platelet sensitivity to agonist stimulation and inhibits thrombosis. Notably, TYMP is an angiogenic factor with the greatest fold increase in patients with type 2 diabetes mellitus, a high-risk cohort for developing severe COVID-19 and it has now been discovered that plasma TYMP levels are significantly increased in COVID-19 patients in an acuity-dependent manner.

In some embodiments of the presently-disclosed subject matter, methods and systems for diagnosis and prognosis of COVID-19 acuity are thus provided that make use of TYMP as a biomarker. In this regard, it should be recognized that the exemplary human biomarkers described herein are not intended to limit the present subject matter to human polypeptide biomarkers or mRNA biomarkers only. Rather, the present subject matter encompasses biomarkers across animal species that are associated with COVID-19.

A “biomarker” is a molecule useful as an indicator of a biologic state in a subject. With reference to the present subject matter, the biomarkers disclosed herein can be polypeptides that exhibit a change in expression level or activity, which can be correlated with the risk of developing, the presence of, or the progression of COVID-19 acuity in a subject. In addition, the biomarkers disclosed herein are inclusive of messenger RNAs (mRNAs) encoding the biomarker polypeptides, as measurement of a change in expression of an mRNA can be correlated with changes in expression of the polypeptide encoded by the mRNA. As such, determining an amount of a biomarker in a biological sample is inclusive of determining an amount of a polypeptide biomarker and/or an amount of an mRNA encoding the polypeptide biomarker either by direct or indirect (e.g., by measure of a complementary DNA (cDNA) synthesized from the mRNA) measure of the mRNA.

In some embodiments of the presently-disclosed subject matter, a method for diagnosing COVID-19 acuity in a subject is provided that includes the steps of: providing a biological sample from the subject; determining an expression level or activity in the sample of TYMP; and comparing the expression level or activity of TYMP in the sample, if present, to a control expression level or activity of TYMP. In some embodiments, the subject is then diagnosed as having COVID-19 acuity or at an increased risk thereof if there is a measurable difference in the expression level or activity of TYMP in the sample as compared to the control level. In some embodiments, the COVID-19 acuity is characterized by thrombosis, inflammation, and/or organ damage in the subject. In some embodiments, a diagnosis of COVID-19 acuity in a subject is made by further determining an amount of c-reactive protein (CRP) and/or lactate dehydrogenase (LDH) in the sample obtained from the subject.

The terms “diagnosing” and “diagnosis” as used herein refer to methods by which the skilled artisan can estimate and even determine whether or not a subject is suffering from a given disease or condition. The skilled artisan often makes a diagnosis on the basis of one or more diagnostic indicators, such as for example a marker, the amount (including presence or absence) of which is indicative of the presence, severity, or absence of the condition.

Along with diagnosis, clinical disease prognosis is also an area of great concern and interest. It is important to know the stage and rapidity of advancement of COVID-19 in order to plan the most effective therapy. If a more accurate prognosis can be made, appropriate therapy, and in some instances less severe therapy for the patient can be chosen. Measurement of biomarker levels disclosed herein can be useful in order to categorize subjects according to advancement of COVID-19 who will benefit from particular therapies and differentiate from other subjects where alternative or additional therapies can be more appropriate.

As such, “making a diagnosis” or “diagnosing”, as used herein, is further inclusive of determining a prognosis, which can provide for predicting a clinical outcome (with or without medical treatment), selecting an appropriate treatment (or whether treatment would be effective), or monitoring a current treatment and potentially changing the treatment, based on the measure of diagnostic biomarker levels disclosed herein.

The phrase “determining a prognosis” as used herein refers to methods by which the skilled artisan can predict the course or outcome of a condition in a subject. The term “prognosis” does not refer to the ability to predict the course or outcome of a condition with 100% accuracy, or even that a given course or outcome is predictably more or less likely to occur based on the presence, absence or levels of test biomarkers. Instead, the skilled artisan will understand that the term “prognosis” refers to an increased probability that a certain course or outcome will occur; that is, that a course or outcome is more likely to occur in a subject exhibiting a given condition, when compared to those individuals not exhibiting the condition. For example, in individuals not exhibiting the condition (e.g., not expressing the biomarker or expressing it at a reduced level), the chance of a given outcome may be about 3%. In certain embodiments, a prognosis is about a 5% chance of a given outcome, about a 7% chance, about a 10% chance, about a 12% chance, about a 15% chance, about a 20% chance, about a 25% chance, about a 30% chance, about a 40% chance, about a 50% chance, about a 60% chance, about a 75% chance, about a 90% chance, or about a 95% chance.

The skilled artisan will understand that associating a prognostic indicator with a predisposition to an adverse outcome is a statistical analysis. For example, a biomarker level (e.g., quantity of expression in a sample) of greater than a control level in some embodiments can signal that a subject is more likely to suffer from or experience COVID-19 acuity than subjects with a level less than or equal to the control level, as determined by a level of statistical significance. Additionally, a change in marker concentration from baseline levels can be reflective of subject prognosis, and the degree of change in marker level can be related to the severity of adverse events. Statistical significance is often determined by comparing two or more populations, and determining a confidence interval and/or a p value. See, e.g., Dowdy and Wearden, Statistics for Research, John Wiley & Sons, New York, 1983, incorporated herein by reference in its entirety. Preferred confidence intervals of the present subject matter are 90%, 95%, 97.5%, 98%, 99%, 99.5%, 99.9% and 99.99%, while preferred p values are 0.1, 0.05, 0.025, 0.02, 0.01, 0.005, 0.001, and 0.0001.

In other embodiments, a threshold degree of change in the level of a prognostic or diagnostic biomarker can be established, and the degree of change in the level of the indicator in a biological sample can simply be compared to the threshold degree of change in the level. A preferred threshold change in the level for markers of the presently disclosed subject matter is about 5%, about 10%, about 15%, about 20%, about 25%, about 30%, about 50%, about 75%, about 100%, and about 150%. In yet other embodiments, a “nomogram” can be established, by which a level of a prognostic or diagnostic indicator can be directly related to an associated disposition towards a given outcome. The skilled artisan is acquainted with the use of such nomograms to relate two numeric values with the understanding that the uncertainty in this measurement is the same as the uncertainty in the marker concentration because individual sample measurements are referenced, not population averages.

In some embodiments of the presently-disclosed subject matter, multiple determination of one or more diagnostic or prognostic biomarkers can be made, and a temporal change in the biomarker can be used to monitor the progression of disease and/or efficacy of appropriate therapies directed against the disease. In such an embodiment for example, one might expect to see a decrease or an increase in the biomarker(s) over time during the course of effective therapy. Thus, the presently disclosed subject matter provides in some embodiments a method for determining treatment efficacy and/or progression of COVID-19 in a subject. In some embodiments, the method comprises determining an amount of TYMP in biological samples collected from the subject at a plurality of different time points and comparing the amounts of TYMP in the samples collected at different time points. For example, a first time point can be selected prior to initiation of a treatment and a second time point can be selected at some time after initiation of the treatment. One or more biomarker levels can be measured in each of the samples taken from different time points and qualitative and/or quantitative differences noted. A change in the amounts of the biomarker levels from the first and second samples can be correlated with determining treatment efficacy and/or progression of the disease in the subject.

The terms “correlated” and “correlating,” as used herein in reference to the use of diagnostic and prognostic biomarkers, refers to comparing the presence or quantity of the biomarker in a subject to its presence or quantity in subjects known to suffer from, or known to be at risk of, a given condition (e.g., COVID-19 acuity); or in subjects known to be free of a given condition, i.e. “normal individuals.” For example, a biomarker level in a biological sample can be compared to a level known to be associated with a specific manifestation of COVID-19 acuity. The sample's biomarker level is said to have been correlated with a diagnosis; that is, the skilled artisan can use the biomarker level to determine whether the subject suffers from or is experiencing a specific manifestation of COVID-19 acuity, and respond accordingly. Alternatively, the sample's biomarker level can be compared to a control marker level known to be associated with a good outcome (e.g., the absence of COVID-19 acuity), such as an average level found in a population of normal subjects.

In certain embodiments, a diagnostic or prognostic biomarker is correlated to a condition or disease by merely its presence or absence. In other embodiments, a threshold level of a diagnostic or prognostic biomarker can be established, and the level of the indicator in a subject sample can simply be compared to the threshold level.

As noted, in some embodiments, multiple determination of one or more diagnostic or prognostic biomarkers can be made, and a temporal change in the marker can be used to determine a diagnosis or prognosis. For example, a diagnostic marker can be determined at an initial time, and again at a second time. In such embodiments, an increase in the marker from the initial time to the second time can be diagnostic of a particular manifestation of COVID-19 acuity, or a given prognosis. Likewise, a decrease in the marker from the initial time to the second time can be indicative of a particular manifestation of COVID-19 acuity, or a given prognosis. Furthermore, in some embodiments, the degree of change of one or more markers can be related to the severity of COVID-19 and future adverse events.

The skilled artisan will understand that, while in certain embodiments comparative measurements can be made of the same diagnostic marker at multiple time points, one can also measure a given marker at one time point, and a second marker at a second time point, and a comparison of these markers can provide diagnostic information.

With regard to the step of providing a biological sample from the subject, the term “biological sample” as used herein refers to any body fluid or tissue potentially comprising the biomarkers. In some embodiments, for example, the biological sample can be a blood sample, a serum sample, a plasma sample, or sub-fractions thereof.

Turning now to the step of identifying an expression level or activity of one or more markers in the biological sample, various methods known to those skilled in the art can be used to identify the one or more markers in the provided biological sample. In some embodiments, determining the amount of biomarkers in samples comprises using a RNA measuring assay to measure miRNA or mRNA encoding biomarker polypeptides in the sample and/or using a protein measuring assay to measure amounts of biomarker polypeptides in the sample.

In certain embodiments, the amounts of biomarkers can be determined by probing for an miRNA or for mRNA of the biomarker in the sample using any RNA identification assay known to those skilled in the art. Briefly, RNA can be extracted from the sample, amplified, converted to cDNA, labeled, and allowed to hybridize with probes of a known sequence, such as known RNA hybridization probes (selective for mRNAs encoding biomarker polypeptides) immobilized on a substrate, e.g., array, or microarray, or quantitated by real time PCR (e.g., quantitative real-time PCR, such as available from Bio-Rad Laboratories, Hercules, California, U.S.A.). Because the probes to which the nucleic acid molecules of the sample are bound are known, the molecules in the sample can be identified. In this regard, DNA probes for one or more biomarkers (e.g., TYMP) can be immobilized on a substrate and provided for use in practicing a method in accordance with the present subject matter.

In some embodiments, determining the amount of biomarkers in samples comprises the use of mass spectrometry and/or immunoassay devices and methods to measure polypeptides in samples, although other methods are well known to those skilled in the art as well. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety. Immunoassay devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, can be employed to determine the presence or amount of analytes without the need for a labeled molecule. See, e.g., U.S. Pat. Nos. 5,631,171; and 5,955,377, each of which is hereby incorporated by reference in its entirety.

Thus, in certain embodiments of the presently-disclosed subject matter, the marker peptides are analyzed using an immunoassay. The presence or amount of a marker (e.g., TYMP) can be determined using antibodies or fragments thereof specific for each marker and detecting specific binding. For example, in some embodiments, the antibody specifically binds TYMP, which is inclusive of antibodies that bind the full-length peptide or a fragment thereof. In some embodiments, the antibody is a monoclonal antibody, such as an anti-TYMP monoclonal antibody. In other embodiments, the antibody is a polyclonal antibody.

Any suitable immunoassay can be utilized, for example, enzyme-linked immunoassays (ELISA), radioimmunoassays (RIAs), competitive binding assays, and the like. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The use of immobilized antibodies or fragments thereof specific for the markers is also contemplated by the presently-disclosed subject matter. The antibodies can be immobilized onto a variety of solid supports, such as magnetic or chromatographic matrix particles, the surface of an assay plate (such as microtiter wells), pieces of a solid substrate material (such as plastic, nylon, paper), and the like. An assay strip can be prepared by coating the antibody or a plurality of antibodies in an array on a solid support. This strip can then be dipped into the test biological sample and then processed quickly through washes and detection steps to generate a measurable signal, such as for example a colored spot.

In some embodiments, mass spectrometry (MS) analysis can be used alone or in combination with other methods (e.g., immunoassays) to determine the presence and/or quantity of the one or more biomarkers of interest in a biological sample. In some embodiments, the MS analysis comprises matrix-assisted laser desorption/ionization (MALDI) time-of-flight (TOF) MS analysis, such as for example direct-spot MALDI-TOF or liquid chromatography MALDI-TOF mass spectrometry analysis. In some embodiments, the MS analysis comprises electrospray ionization (ESI) MS, such as for example liquid chromatography (LC) ESI-MS. Mass analysis can be accomplished using commercially-available spectrometers, such as for example triple quadrupole mass spectrometers. Methods for utilizing MS analysis, including MALDI-TOF MS and ESI-MS, to detect the presence and quantity of biomarker peptides in biological samples are known in the art. See for example U.S. Pat. Nos. 6,925,389; 6,989,100; and 6,890,763 for further guidance, each of which is incorporated herein by this reference.

With further respect to the measurement of the biomarkers described herein, in some embodiments, the TYMP biomarker is detected in the sample using a method selected from the group consisting of ELISA, Luminex, Western blot, dot blot, immunoprecipitation, immunohistochemistry, immunocytochemistry, immunofluorescence, immunodetection methods, optical spectroscopy, radioimmunoassay, mass spectrometry, HPLC, qPCR, RT-qPCR, multiplex qPCR, SAGE, RNA-seq, microarray analysis, FISH, MassARRAY technique, and combinations thereof.

Although certain embodiments of the methods only call for a qualitative assessment of the presence or absence of the one or more markers in the biological sample, other embodiments of the methods call for a quantitative assessment of the amount of each of the one or more markers in the biological sample. Such quantitative assessments can be made, for example, using one of the above mentioned methods, as will be understood by those skilled in the art.

In certain embodiments of the methods described herein, it can be desirable to include a control sample that is analyzed concurrently with the biological sample, such that the results obtained from the biological sample can be compared to the results obtained from the control sample. Additionally, it is contemplated that standard curves can be provided, with which assay results for the biological sample can be compared. Such standard curves present levels of biomarkers as a function of assay units, i.e., fluorescent signal intensity, if a fluorescent signal is used. Using samples taken from multiple donors, standard curves can be provided for control levels of the one or more markers in normal tissue.

The analysis of markers can be carried out separately or simultaneously with additional markers within one test sample. For example, several markers can be combined into one test for efficient processing of a multiple samples and for potentially providing greater diagnostic and/or prognostic accuracy. In addition, one skilled in the art would recognize the value of testing multiple samples (for example, at successive time points) from the same subject. Such testing of serial samples can allow the identification of changes in marker levels over time. Increases or decreases in marker levels, as well as the absence of change in marker levels, can provide useful information about the disease status that includes, but is not limited to, identifying the approximate time from onset of the event, the presence and amount of salvageable tissue, the appropriateness of drug therapies, the effectiveness of various therapies, and identification of the subject's outcome, including risk of future events.

The analysis of markers can be carried out in a variety of physical formats as well. For example, the use of microtiter plates or automation can be used to facilitate the processing of large numbers of test samples. Alternatively, single sample formats could be developed to facilitate immediate treatment and diagnosis in a timely fashion, for example, in ambulatory transport or emergency room settings.

As mentioned above, depending on the embodiment of the method, identification of the one or more markers can be a qualitative determination of the presence or absence of the markers, or it can be a quantitative determination of the concentration of the markers. In this regard, in some embodiments, the step of identifying the subject as having COVID-19 acuity or a risk thereof requires that certain threshold measurements are made, i.e., the levels of the one or more markers in the biological sample exceed control level. In certain embodiments of the method, the control level is any detectable level of the marker. In other embodiments of the method where a control sample is tested concurrently with the biological sample, the control level is the level of detection in the control sample. In other embodiments of the method, the control level is based upon and/or identified by a standard curve. In other embodiments of the method, the control level is a specifically identified concentration, or concentration range. As such, the control level can be chosen, within acceptable limits that will be apparent to those skilled in the art, based in part on the embodiment of the method being practiced and the desired specificity, etc.

In some embodiments of the presently-disclosed subject matter, a system, kit, or assay for diagnosing COVID-19 acuity in a subject is provided, or a system, kit, or assay for determining whether to initiate or continue prophylaxis or treatment of COVID-19 in a subject is provided. Such systems, kits, or assays can be provided, for example, as commercial kits that can be used to test a biological sample, or series of biological samples, from a subject. The system can also include certain samples for use as controls. The system can further include one or more standard curves providing levels of markers as a function of assay units.

In some embodiments, a system for the analysis of biomarkers is provided that comprises antibodies having specificity for one or more markers associated with COVID-19 acuity. Such a system can comprise devices and reagents for the analysis of at least one test sample. The system can further comprise instructions for using the system and conducting the analysis. Optionally the systems can contain one or more reagents or devices for converting a marker level to a diagnosis or prognosis of the subject.

Further provided, in some embodiments, is a method for screening for a compound useful for treating COVID-19, comprising the steps of: contacting a cell with an effective amount of a test compound; and detecting an expression level or activity level of thymidine phosphorylase in the cell in the presence of the test compound. In some embodiments, and similar to the assays and systems described above, the screening methods can further make use of a control sample as would be recognized by those skilled in the art. In some embodiments of the screening methods, the test compound is identified as a compound useful for treating COVID-19 if there is a measureable difference in the expression level or activity level of thymidine phosphorylase subsequent to contacting the cell with the test compound.

Still further provided, in some embodiments of the presently-disclosed subject matter, are methods for treating COVID-19 in a subject. In some embodiments of the presently-disclosed subject matter, a method of treating COVID-19 is provided that comprises reducing an expression level or activity of TYMP in a subject in need thereof. In some embodiments of the diagnostic and therapeutic methods described herein, the diagnosis and treatment of COVID-19 is inclusive of diagnosis and treatment of a viral infection caused by a wild-type SARS-CoV-2 virus as well as variants of such as virus as both the wild-type virus and the variants identified to date have been shown to bind to ACE2 as a receptor for the viral particles. Moreover, and without wishing to be bound by any particular theory or mechanism, it is believed that the diagnostic and therapeutic methods described herein are further applicable to infection with a SARS-CoV virus as SARS-CoV also makes use of ACE2 as receptor to infect a host.

In some embodiments of the therapeutic methods of the presently-disclosed subject matter, reducing the expression level or activity of TYMP comprises administering to the subject a TYMP inhibitor. In some embodiments of the therapeutic methods, administering the effective amount of the TYMP inhibitor reduces an amount of platelet activation and aggregation. In some embodiments, administering the effective amount of the TYMP inhibitor reduces an amount of inflammation, D-dimer formation, organ damage, and/or thrombosis in the subject.

With respect to the TYMP inhibitors that can be utilized, in some embodiments, the inhibitor utilized is a genetic inhibitor such as siRNA, miRNA, shRNA, CRISPR, small molecule inhibitors, peptide or protein inhibitors including antibodies. In some embodiments, the TYMP inhibitor is tipiracil, whose chemical structure is provided below. See also https://pubchem.ncbi.nlm.nih.gov/compound/Tipiracil, which is incorporated herein by reference.

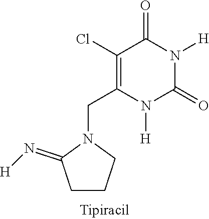 [SMILES: [H]/N=C1\CCCN1Cc1[nH]c(=O)[nH]c(=O)c1Cl]

With respect to the treatment of COVID-19 described herein, the terms “treatment” or “treating” are used herein to refer any treatment of COVID-19, including but not limited to prophylactic treatment and therapeutic treatment. As such, the terms “treatment” or “treating” include, but are not limited to: reducing the development or likelihood of development of a COVID-19; inhibiting the progression of COVID-19; arresting or reducing the further development of COVID-19; reducing the severity of COVID-19; ameliorating or relieving symptoms associated with COVID-19; and causing a regression of COVID-19 or one or more of the symptoms associated with COVID-19.

For administration of a therapeutic composition as disclosed herein (e.g., a TYMP inhibitor), conventional methods of extrapolating human dosage based on doses administered to a murine animal model can be carried out using the conversion factor for converting the mouse dosage to human dosage: Dose Human per kg=Dose Mouse per kg/12 (Freireich, et al., (1966) Cancer Chemother Rep. 50: 219-244). Doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich, et al. (Freireich et al., (1966) Cancer Chemother Rep. 50:219-244). Briefly, to express a mg/kg dose in any given species as the equivalent mg/sq m dose, multiply the dose by the appropriate km factor. In an adult human, 100 mg/kg is equivalent to 100 mg/kg×37 kg/sq m=3700 mg/m². In some embodiments that make use of a TYMP inhibitor, such as tipiracil, the inhibitor is administered to the subject at a dose of about 1 mg/kg/day.

Suitable methods for administering a therapeutic composition in accordance with the methods of the presently-disclosed subject matter include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for attenuation or reduction of a disease or condition or one or more associated symptoms.

Regardless of the route of administration, the inhibitors utilized in accordance with the presently-disclosed subject matter are typically administered in an amount effective to achieve the desired response. As such, the term “effective amount” is used herein to refer to an amount of the therapeutic composition (e.g., a TYMP inhibitor and a pharmaceutically vehicle, carrier, or excipient) sufficient to produce a measurable biological response (e.g., a decrease in inflammation or organ damage). Actual dosage levels of active ingredients in a therapeutic composition of the presently-disclosed subject matter can be varied so as to administer an amount of the active compound(s) that is effective to achieve the desired therapeutic response for a particular subject and/or application. Of course, the effective amount in any particular case will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

In some embodiments of the therapeutic methods for the treatment of COVID-19, the therapeutic agents used in accordance with the presently-disclosed subject matter (e.g., the TYMP inhibitors such as tipiracil) can be used alone or in combination with other therapeutic agents capable of treating COVID-19. In some embodiments, administration of a TYMP inhibitor in combination with other therapeutic agents allows for a synergistic effect and/or allows for a lower of dose of each agent to be utilized relative to the dose that would should be required should a single therapeutic agent be administered by itself. For instance, in some embodiments, tipiracil can be administered alone or in combination with other therapeutic agents, such as, in certain embodiments, an effective amount of dexamethasone or another corticosteroid for the treatment of COVID-19.

For additional guidance regarding formulation and dose, see U.S. Pat. Nos. 5,326,902; 5,234,933; PCT International Publication No. WO 93/25521; Berkow et al., (1997) The Merck Manual of Medical Information, Home ed. Merck Research Laboratories, Whitehouse Station, New Jersey; Goodman et al., (1996) Goodman & Gilman's the Pharmacological Basis of Therapeutics, 9th ed. McGraw-Hill Health Professions Division, New York; Ebadi, (1998) CRC Desk Reference of Clinical Pharmacology. CRC Press, Boca Raton, Florida; Katzung, (2001) Basic & Clinical Pharmacology, 8th ed. Lange Medical Books/McGraw-Hill Medical Pub. Division, New York; Remington et al., (1975) Remington's Pharmaceutical Sciences, 15th ed. Mack Pub. Co., Easton, Pennsylvania; and Speight et al., (1997) Avery's Drug Treatment: A Guide to the Properties, Choice, Therapeutic Use and Economic Value of Drugs in Disease Management, 4th ed. Adis International, Auckland/Philadelphia; Duch et al., (1998) Toxicol. Lett. 100-101:255-263.

As noted, in some embodiments of the presently-disclosed subject matter, reducing the expression level or activity of TYMP, such as through the administration of a TYMP inhibitor, reduces one or more factors and/or symptoms associated with COVID-19 an amount of inflammation, D-dimer formation, organ damage, and/or thrombosis in a subject. Various methods known to those skilled in the art can be used to determine an increase or a reduction in such factors and symptoms associated with COVID-19 in a subject. For example, in certain embodiments, the amounts of expression of an inflammation marker (e.g., an inflammatory cytokine) in a subject can be determined by probing for mRNA or protein of the inflammation marker in a biological sample obtained from the subject (e.g., a tissue sample, a urine sample, a saliva sample, a blood sample, a serum sample, a plasma sample, or sub-fractions thereof) using any RNA or protein identification assay known to those skilled in the art.

As used herein, the term “subject” includes both human and animal subjects. Thus, veterinary therapeutic uses are provided in accordance with the presently disclosed subject matter. As such, the presently-disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like. In some embodiments, the subject is a male subject.

The practice of the presently-disclosed subject matter can employ, unless otherwise indicated, conventional techniques of cell biology, cell culture, molecular biology, transgenic biology, microbiology, recombinant DNA, and immunology, which are within the skill of the art. Such techniques are explained fully in the literature. See e.g., Molecular Cloning A Laboratory Manual (1989), 2nd Ed., ed. by Sambrook, Fritsch and Maniatis, eds., Cold Spring Harbor Laboratory Press, Chapters 16 and 17; U.S. Pat. No. 4,683,195; DNA Cloning, Volumes I and II, Glover, ed., 1985; Oligonucleotide Synthesis, M. J. Gait, ed., 1984; Nucleic Acid Hybridization, D. Hames & S. J. Higgins, eds., 1984; Transcription and Translation, B. D. Hames & S. J. Higgins, eds., 1984; Culture Of Animal Cells, R. I. Freshney, Alan R. Liss, Inc., 1987; Immobilized Cells And Enzymes, IRL Press, 1986; Perbal (1984), A Practical Guide To Molecular Cloning; See Methods In Enzymology (Academic Press, Inc., N.Y.); Gene Transfer Vectors For Mammalian Cells, J. H. Miller and M. P. Calos, eds., Cold Spring Harbor Laboratory, 1987; Methods In Enzymology, Vols. 154 and 155, Wu et al., eds., Academic Press Inc., N.Y.; Immunochemical Methods In Cell And Molecular Biology (Mayer and Walker, eds., Academic Press, London, 1987; Handbook Of Experimental Immunology, Volumes I-IV, D. M. Weir and C. C. Blackwell, eds., 1986.

The presently-disclosed subject matter is further illustrated by the following specific but non-limiting examples.

### EXAMPLES

SARS-CoV-2 uses the angiotensin-converting enzyme (ACE) 2 to enter host cells. The binding of SARS-CoV-2 to ACE2 is mediated by the coronavirus spike protein. How the SARS-CoV-2 spike protein affected host cells and ACE2 function, however, was not clear. Thymidine phosphorylase (TYMP), on the other hand, catalyzes the reversible conversion of thymidine to thymine and 2-deoxy-D-ribose-1 phosphate, and plays important roles in enhancing inflammatory cytokine expression, platelet signaling activation, and thrombosis. As described below, using the MGH (Massachusetts General Hospital) Emergency Department COVID-19 Cohort with Olink Proteomics, it was found that TYMP levels in COVID-19 patients increased in an acuity-dependent manner. Plasma TYMP increased earlier than C-reactive protein (CRP), an inflammation marker, and was positively associated with levels of organ and lung injury markers. This finding was supported by another gene expression study using platelets harvested from COVID-19 patients, in which platelet TYMP mRNA increased 1.8-log 2 fold (3.5-fold) in patients admitted to the intensive care unit (ICU) but only 1.45-log 2 fold (2.7-fold) in non-ICU patients compared to healthy controls. Significantly, treating BEAS-2B, a human bronchial epithelial cell line, with spike protein- or its receptor-binding domain-containing COS-7 cell lysate, dramatically increased TYMP expression and activation of NF-κB, a transcription factor that is important for inflammatory responses. Moreover, it was found that TYMP was highly expressed in upper airway epithelial cells as well as type II alveolar epithelial cells, which are the primary host cells mediating SARS-CoV-2 entry. Treating human ACE2 transgenic mice [B6.Cg-Tg(K18-ACE2)2Prlmn/J] with spike protein markedly enhanced thrombosis, which was then significantly inhibited by tipiracil (1 mg/kg/day), a potent and selective TYMP inhibitor. Taken together, the studies described herein demonstrated that TYMP was significantly increased in the COVID-19 milieu. Accordingly, and without wishing to be bound by any particular theory or mechanism, it was believed that increased TYMP expression induced by SARS-CoV-2 spike protein plays an important mechanistic role in the development of severe COVID-19 (FIG. 1).

### Example 1—TYMP Expression is Increased in Patients with COVID-19 in an Acuity-Dependent Manner

The database provided by the Massachusetts General Hospital (MGH) emergency department COVID-19 Cohort (Filbin, Goldberg, Hacohen) with Olink Proteomics was utilized, which includes four 384-plex panels focused on inflammation, oncology, cardiometabolic, and neurology proteins. TYMP was only found in the cardiometabolic panel. A total of 733 data points, including day 0 (358), day 3 (202), day 7 (131), and death (42) were extracted. The TYMP data were sorted based on the final diagnoses: COVID-19 negative or positive. Both groups were further sorted based on age, BMI (body mass index), days after hospitalization, acuity assessed based on WHO Ordinal Outcomes Score, levels of plasma D-dimer, absolute counts of nucleated cells, plasma CRP (C-reactive protein) and LDH (lactate dehydrogenase) levels, as well as fever. The data were also sorted based on the presence or absence of pre-existing disease and respiratory symptoms, including sore throat, congestion, productive or dry cough, shortness of breath or hypoxia, or chest pain. TYMP expression was then analyzed and compared among the defined groups.

To further examine the potential role of TYMP in virus infection, the Gene Expression Omnibus (GEO) profile was searched using key words “TYMP+virus”. TYMP expression was extracted and analyzed in H1N1 infected bronchia epithelial cells (GDS4855) and Sendai virus infected monocytes (GSE67198). TYMP expression was also analyzed in the IFN-γ, which was upregulated by SARS-CoV-2 infection, treated primary cultured bronchial epithelial cells (GDS1256).

The data were analyzed using the 2-tailed Student's t test with GraphPad Prism (version 9.0.0). Data were expressed as mean±SD. The data was analyzed for normality and equal variance as a justification for using the 2-tailed Student's t test. A p≤0.05 was considered statistically significant.

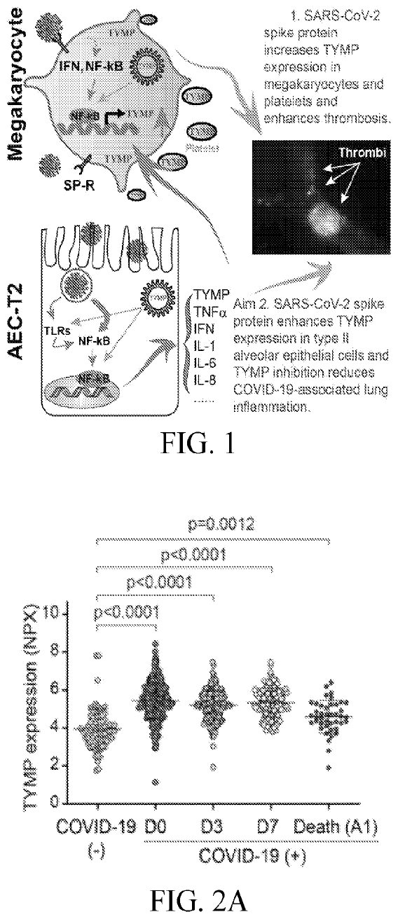

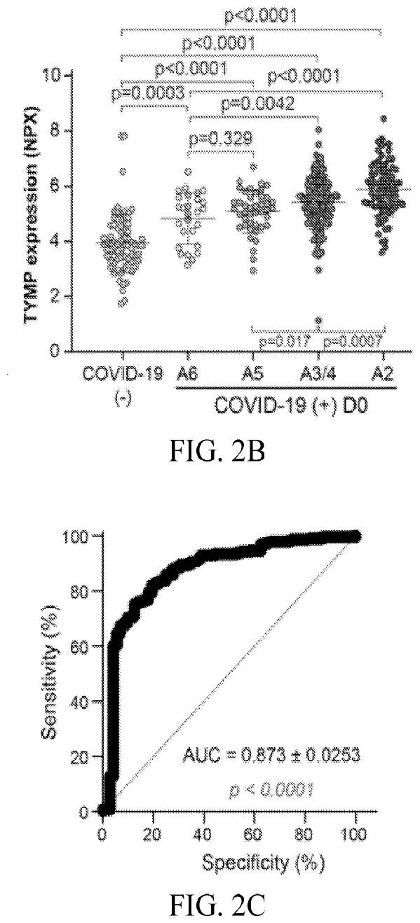

In the MGH study, TYMP was only found in the cardiometabolic panel, and 733 TYMP data points were extracted. Among the MGH cohort, 78 patients were negative for SARS-CoV-2, and 306 were positive. In particular, this database contained data from samples collected from 358 patients (285 COVID-19) on day 0, 202 patients (198 COVID-19) on day 3, 131 patients (all COVID-19) on day 7, and 42 patients who died from COVID-19 within 28 days. It was found that plasma TYMP levels were significantly increased in COVID-19 patients on days 0, 3, and 7 compared to non-COVID-19 patients (FIG. 2A). Patients who died from COVID-19 (A1 in FIG. 2A) also had higher plasma TYMP levels. By stratifying patients by acuity scores based on the WHO Ordinal Outcomes Score, it was found that TYMP was increased in COVID-19 patients in an acuity-dependent manner on day 0 (FIG. 2B). Compared to non-COVID-19 patients, COVID-19 patients had significantly higher plasma TYMP levels, even those discharged from the Emergency Department (A6) (p=0.0003, FIG. 2B). Among hospitalized patients, the lowest plasma TYMP levels were observed in patients who did not need supplemental oxygen (A5), middle levels were found in patients who needed oxygen inhalation (A3/4), and intubated patients (A2) had the highest plasma TYMP levels. On day 3, intubated patients still had higher plasma TYMP levels than patients without supplementary oxygen. These findings are in line with a platelet gene expression study involving COVID-19 patients which showed that TYMP mRNA levels increased 1.64-log 2 fold (3.1 fold change) compared to healthy volunteers. TYMP levels increased 1.83-log 2 fold (3.6 fold change) in ICU patients and 1.45 log 2 fold (2.7 fold change) in non-ICU patients, further showed an acuity-dependent manner. To determine the diagnostic value of TYMP in COVID-19, Receiver Operating Characteristic (ROC) analysis was conducted based on TYMP plasma levels on Day 0. ROC analysis has been used in many diverse fields to evaluate diagnostic testing performance and is widely used by Clinicians. The 73 COVID-19 negative patients were used as controls, and the 285 COVID-19 positive individuals were used as patients. As shown in the FIG. 2C, the AUC is 0.873, indicating that TYMP is a very sensitive and specific marker in diagnosing severe COVID-19. Taken together, it was found that TYMP levels are increased in COVID-19 patients, and TYMP is a novel acuity marker of COVID-19.

### Example 2—TYMP Expression is Positively Correlated with COVID-19 Severity Markers

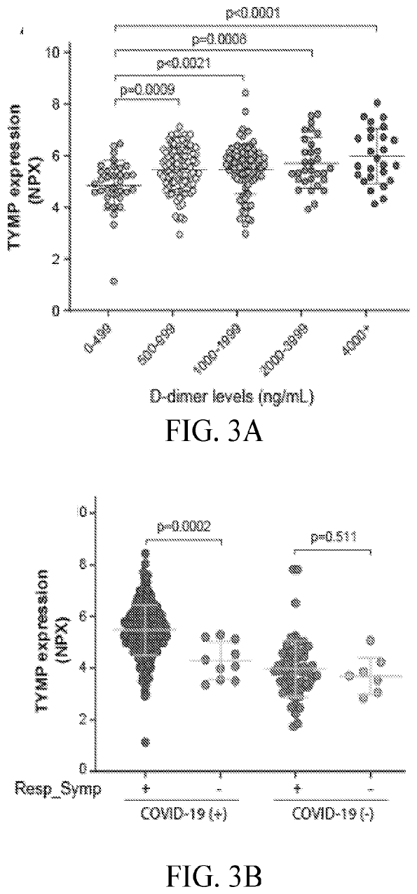

COVID-19 patients have reduced platelet counts and elevated plasma D-dimer, an indicator of a thrombotic event. Because TYMP plays a mechanistic role in platelet activation and thrombosis, the relationship between TYMP and plasma D-dimer levels was analyzed. It was found that the higher the patient D-dimer concentration, the higher the plasma TYMP level (FIG. 3A). These data indicated that TYMP is correlated with a COVID-19-associated thrombotic event. TYMP expression was not associated with monocyte, lymphocyte, or neutrophil counts and fever. Moreover, age, BMI, primary kidney disease, hypertension, pre-existing immunocompromised conditions, pre-existing gastrointestinal diseases, and diabetes also did not affect TYMP expression levels. TYMP expression levels were similar between COVID-19-negative patients without and with respiratory symptoms, including sore throat, congestion, productive or dry cough, shortness of breath or hypoxia, or chest pain. However, TYMP expression was significantly increased in COVID-19 patients with respiratory symptoms compared to COVID-19 patients without respiratory symptoms (FIG. 3B). These data indicated that plasma TYMP levels also reflect the severity of COVID-19-associated lung injury in addition to thrombotic events.

### Example 3—TYMP Expression is Positively Correlated with COVID-19-Associated Inflammation and Organ Damage

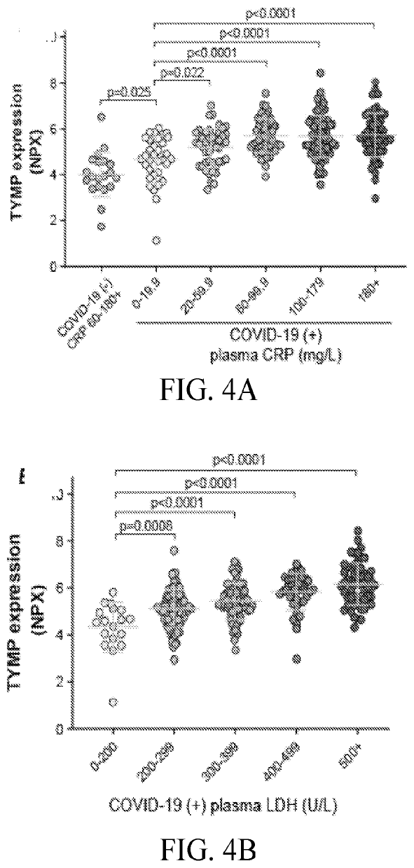

CRP is a critical component of the immune system and a predictive factor for inflammation and future risk of cardiovascular events. It was found that patients with higher CRP levels also had higher plasma TYMP levels (FIG. 4A). Because patients with the lowest CRP levels (<20 mg/L) already had significantly higher TYMP expression than non-COVID-19 patients, even if they had a very high level of plasma CRP (>60-180 mg/L, FIG. 4A, p=0.025), this finding further indicated that TYMP is a more sensitive and specific marker for COVID-19 severity. Consequently, it was found that TYMP expression was significantly correlated with LDH plasma levels, an indicator of tissue damage (FIG. 4B).

### Example 4—Spike Protein (SP) and its Receptor Binding Domain (RBD) Upregulate TYMP Expression in Human Bronchial Epithelial Cells, and Inhibition of TYMP Dramatically Inhibited SP-Enhanced Thrombosis In Vivo

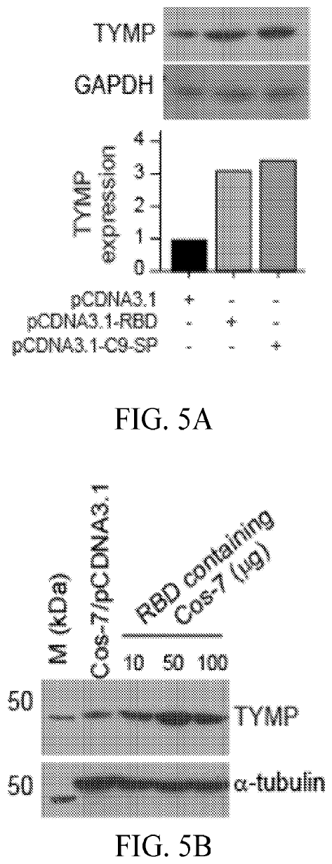

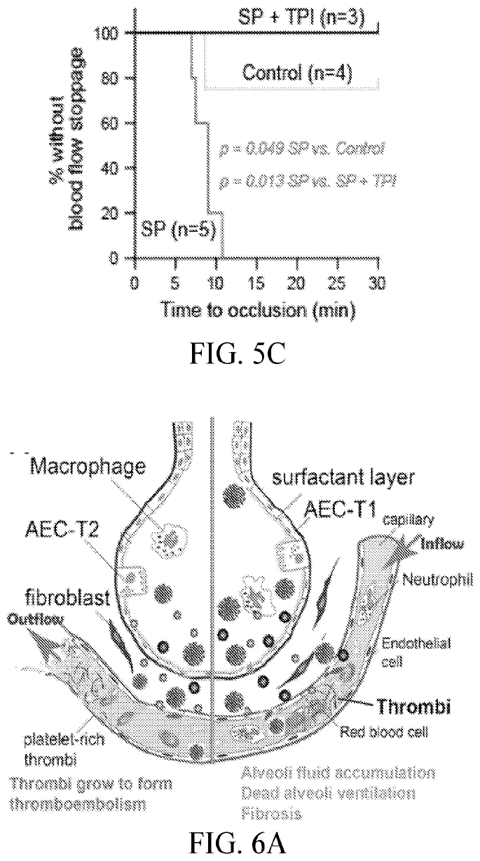

To examine how SARS-CoV-2 infection increases TYMP expression, human normal bronchial epithelium, BEAS-2B cells (CRL-9609™, ATCC), were treated with cell lysates prepared from COS-7 cells (CRL-1651™, ATCC) transfected with pCDNA3.1, pCDNA3.1/GFP-RBD (pcDNA3-SARS-CoV-2-S-RBD-sfGFP, addgene, item #141184), or pCDNA3.1/C9-SP plasmid (pcDNA3.1-SARS2-Spike, addgene, item #145032). As shown in FIGS. 5A and 5B, both RBD and SP containing COS-7 lysates significantly increased TYMP expression. RBD dose-dependently increased TYMP expression, indicating that binding of SARS-CoV-2 SP to its receptor on BEAS-2B is sufficient to change host cell gene expression.

As mentioned above, it is appreciated that ACE2 activation promotes an antithrombotic effect. Since mouse ACE2 does not avidly bind the SARS-CoV-2 SP, and wild-type (WT) C57BL/6J mice do not develop severe illness, ACE2^{TG} mice were purchased from the Jackson Laboratory (B6.Cg-Tg(K18-ACE2)2Prlmn/J, Strain #034860). This mouse strain was susceptible to SARS-CoV-2 infection and partially recapitulates human COVID-19, and has been considered as one of the best available models for COVID-19 study. Recent studies have also indicated that this model recapitulates activation of coagulation, complement, and interferon responses in circulating platelets, which provides valuable insight into platelet pathology in the COVID-19 milieu. Compared to normal C57BL/6J mice, the ACE2^{TG} mice are antithrombotic as no occlusive thrombosis formation was observed when subjecting these mice to the 7.5% FeCl₃ injury-induced carotid artery thrombosis model (data not shown). Intraperitoneal administration of 500 μg of COS-7 cell lysate prepared from the pCDNA3.1-transfected cells did not affect in vivo thrombosis examined 3 days post injection in compared to the untreated animals (FIG. 5C, green line). However, injection of cell lysate containing SP dramatically promoted thrombosis (FIG. 5C, red line). The SP-enhanced thrombosis was completely inhibited by tipiracil (FIG. 5C, black line). These data indicated that binding of SP to host cells increased TYMP expression and TYMP inhibition can attenuate the SP-enhanced thrombosis.

### Example 5—SARS-CoV-2 Spike Protein Enhances TYMP Expression in Type II Alveolar Epithelial Cells and TYMP Inhibition Reduces COVID-19-Associated Lung Inflammation

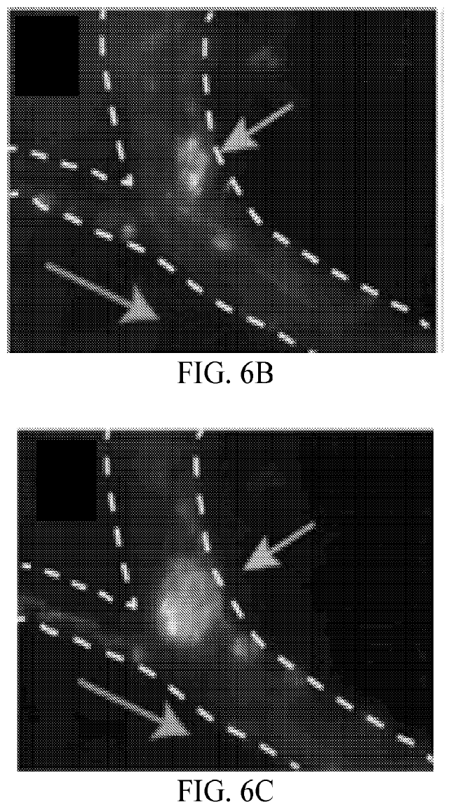

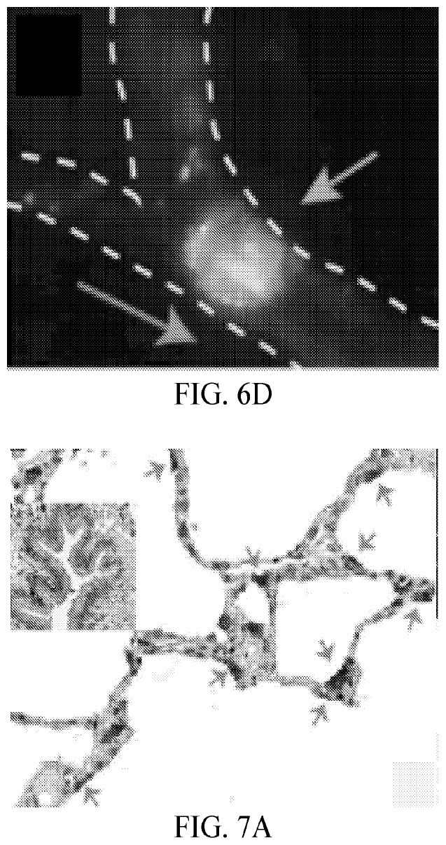

The most severe complication of COVID-19 is acute respiratory distress syndrome (ARDS), which is driven by diffuse alveolar damage caused by “cytokine storm” syndrome. Autopsy studies have found the presence of inflammation and thrombosis, suggesting microvascular immunothrombi formation, also known as inflammatory thrombosis. As shown in FIG. 6A, there are two different alveolar epithelial cell types: type I alveolar epithelial cell (AEC-T1) and type II alveolar epithelial cell (AEC-T2). AEC-T2 cells have fundamental functions, including producing all of the components of the surfactant complex, immune defense, and preventing alveolar collapse. ACE2 and TMPRSS2 are co-expressed in AEC-T2 cells, suggesting that AEC-T2 cells may play a role in developing severe COVID-19. Several autopsy studies also found that AEC-T2 cells are hyperplastic, further suggesting that it is associated with the acute lung injury observed in COVID-19.

TYMP may participate in SARS-CoV-2-induced inflammation. Several inflammatory cytokines, including TNFα, IL-1, IL-6, IL-8, IL-17, IFN-γ, and G-CSF, can induce TYMP expression. TYMP also enhances the expression of some inflammatory cytokines, including IL-8 and CXCL10. All these cytokines are components of the inflammatory “cytokine storm” associated with COVID-19. In addition to their hemostatic effects, platelets are key mediators of inflammation and act as infectious agent sensors. By interaction with cell surface receptors and pathogens (pathogen pattern recognition receptors) or immune system derivatives (immunoglobulin Fc and complement receptors), activated platelets can interact with white blood cells to facilitate pathogen clearance through clot formation. However, the interactions between platelets and neutrophils or other nucleated cells can form immunothrombi/microthrombi (FIG. 6B-6D) that could potentially block blood flow in alveolar capillaries, leading to reduced or dead alveolar ventilation. As shown in FIG. 6A, alveoli are accompanied by pulmonary capillaries. The alveolocapillary membrane is made up of alveolar epithelium, capillary endothelium, and their fused basement membranes. This membrane is water-like and allows the free exchange of viruses, cytokines, and chemokines. Thus, SARS-CoV-2 can pass through the alveolocapillary membrane to infect the pulmonary capillary endothelium. In this regard, and again without wishing to be bound by any particular theory or mechanism, it was believed that TYMP participates in SARS-CoV-2-induced cytokine generation in bronchial epithelium, AEC-T2 cells, and pulmonary capillary endothelium, contributing to immunothrombi formation. If a thrombus forms on the outflow side of the capillary, it may dissociate as an embolus from its original site, resulting in a thromboembolism in the arterial system, as the thrombus grows at the site where outflow capillaries merge into bigger vessels (FIG. 6B-D). This phenomenon can provide a mechanism for the diffuse microthrombi found in the major organs, lower limbs, hands, and mesenteric arteries of COVID-19 patients.

### Example 6—TYMP is Expressed in AEC-T2 and Bronchia Epithelial Cells and Increased in the Inflammatory Lungs from an Asthma Mouse Model

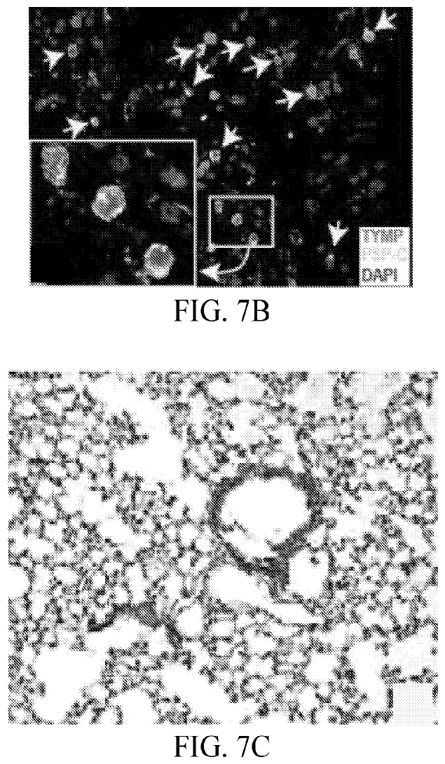

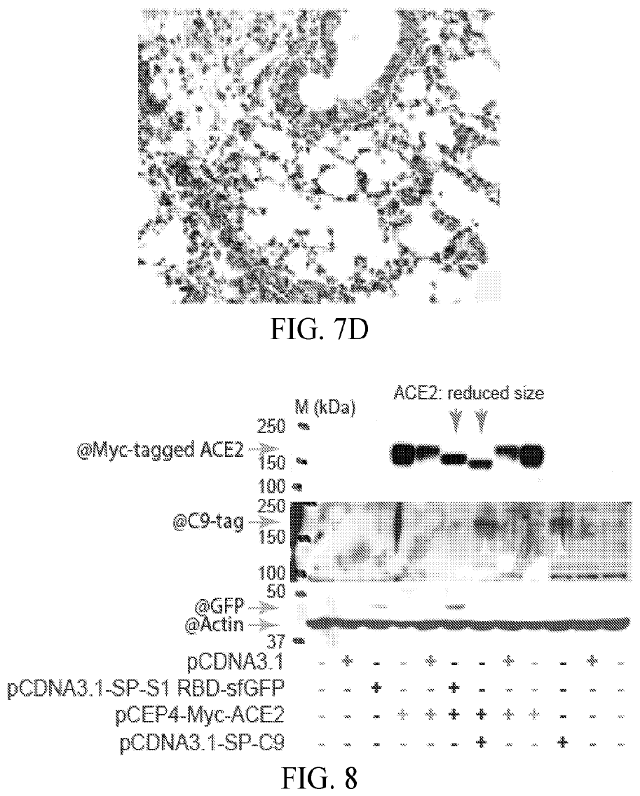

Using data from “The Human Protein Atlas,” it was found that TYMP is expressed in AEC-T2 cells and bronchial epithelium (FIG. 7A). This observation was confirmed by a pathologist from the Marshall University Joan C. Edwards School of Medicine. The staining pattern was more similar to AEC-T2-specific staining observed in another Human Protein Atlas study (HPA010928) than macrophage staining, which comprises most of the alveolar lumen (data not shown). This finding was further confirmed by double staining for TYMP and prosurfactant protein C (PSP-C), an AEC-T2 specific marker (FIG. 7B). TYMP expression is dramatically increased in COVID-19 patients with respiratory symptoms (FIG. 3B), indicating that SARS-CoV-2-induced increase of TYMP expression may result in lung injury. To confirm this hypothesis, TYMP levels in mouse lung harvested from ovalbumin-induced asthmatic mice were determined by immunohistochemical staining. As shown in FIGS. 7C and 7D, TYMP expression was dramatically increased in lung of asthmatic mice compared to normal mouse lung. These data suggest that TYMP participates in lung reactions in response to inflammatory stimulation.

### Example 7—SARS-CoV-2 SP and RBD Lead to ACE2 Reduced Sizes

ACE2 may protect the lungs as the administration of the ACE2 enzymatic product Ang1-7 attenuates airway remodeling and infiltration of inflammatory cells into allergic asthmatic lungs. SARS-CoV infection reduces ACE2 expression in lung cells, binding of SARS-CoV, SARS-CoV-2 or purified SP induces ACE2 endocytosis, suggesting that this type of virus could enhance inflammation and lung injury through ACE2 down regulation. To test this hypothesis, plasmids encoding N-terminal myc-tagged human ACE2 (pCEP4-myc-ACE2, addgene, item #141185) were co-transfected with C-9-tagged full-length SARS-CoV-2 SP or GFP-tagged 51 RBD domain into COS-7 cells and ACE2 expression was examined with an anti-myc antibody. As shown in FIG. 8, co-expression of ACE2 with SP or RBD led to ACE2 changing to two smaller sizes. These data indicated that SP and RBD may enhance lung injury and inflammation through modification of ACE2 function.

### Example 8—SARS-CoV-2 SP Binding Leads to Formation of a Positive Feedback Loop Between TYMP and NF-κB Activation in Host Cells

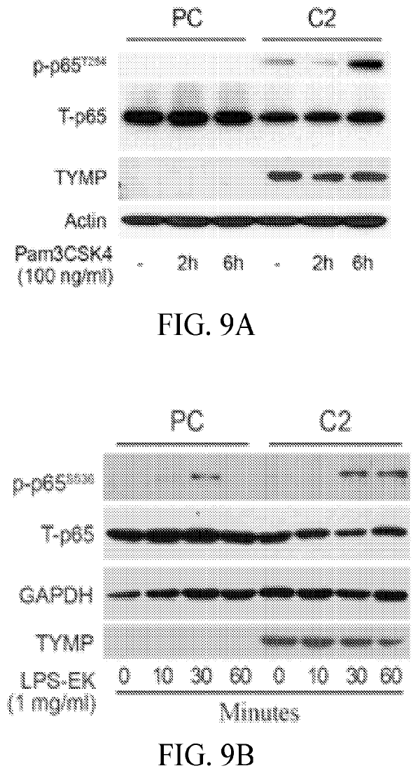

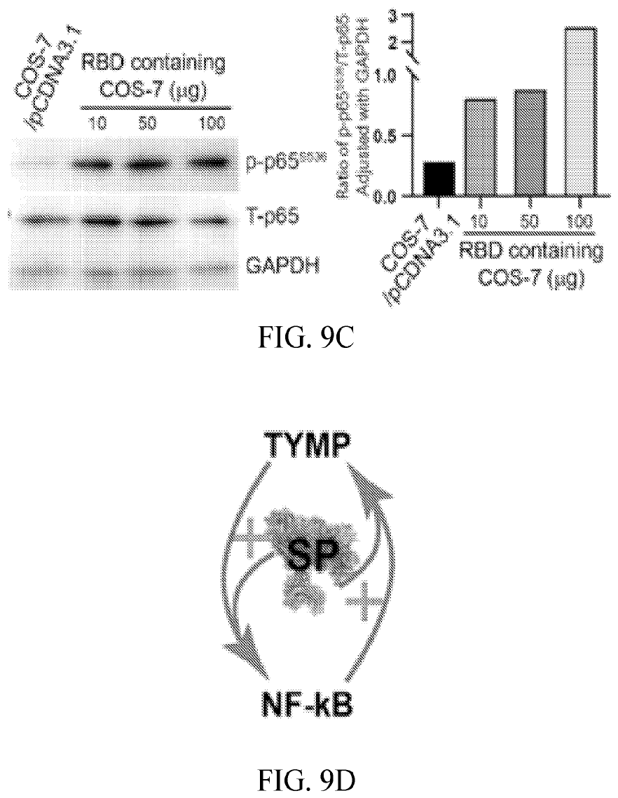

One of the major cellular responses after coronavirus infection is the activation of NF-κB signaling. Toll-like receptors (TLRs) recognize pathogen-associated molecular patterns and danger-associated molecular patterns and induce intracellular signaling cascade activation, especially NF-κB activation, to eliminate pathogens through the production of proinflammatory cytokines. The human TYMP gene was previously transfected into rat vascular smooth muscle cells (VSMCs) and a stable TYMP-overexpressing VSMC cell line, C2, was established. Empty vector transfected VSMCs, PC, were used as control. To determine whether TYMP is involved in TLR signaling, serum-starved C2 and PC cells were stimulated with Pam3CSK4, a TLR1/2 signaling agonist, or LPS-EK, a TLR4 agonist, for the indicated times (FIGS. 9A and 9B). TLR ligands were purchased from InvivoGen (Cat #: tlrl-kit1mw, Mouse TLR1-9 Agonist kit). Activation of NF-κB was examined by western blot of p65 phosphorylation at T254 (p-p65^{T254}) or S536 (p-p65^{S536}). Pam3CSK4 dramatically increased NF-κB activation in C2, but not in PC cells (FIG. 9A). TYMP overexpression enhanced NF-κB p65 phosphorylation at T254 even in serum-free condition without Pam3CSK4 stimulation. These data indicated that increase of TYMP results in a constitutive NF-κB activation. Phosphorylation of T254 leads to Pin1-dependent prolyl isomerization of p65, which increases p65 stability, nuclear accumulation, and enhances transcription. LPS-EK also significantly increased and prolonged NF-κB p65 phosphorylation at S536 (FIG. 9B). By using samples for FIG. 5B, it was found that treating BEAS-2B with RBD also significantly enhanced NF-κB activation (FIG. 9C). In combination with data shown in FIGS. 5A and 5B, in which it was found that SP and RBD enhanced TYMP expression in BEAS-2B cells, these data indicated that SP/RBD-increased TYMP expression would be essential for TLR signaling transduction and constitutive NF-κB activation after SARS-CoV-2 infection. TYMP is NF-κB-dependently increased in astrocyte and plays an important role in promoting inflammation in the central nervous system. Taken together, the findings indicate a positive feedback loop between TYMP and NF-κB (FIG. 9D), which will aggravate inflammation and lead to “cytokine storm” in the COVID-19 milieu.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Zhu N, Zhang D, Wang W, et al. A Novel Coronavirus from Patients with Pneumonia in China, 2019. N Engl J Med. 2020; 382(8):727-733.
2. Jackson C B, Farzan M, Chen B, Choe H. Mechanisms of SARS-CoV-2 entry into cells. Nature Reviews Molecular Cell Biology. 2021.
3. Wu Z, McGoogan J M. Characteristics of and Important Lessons From the Coronavirus Disease 2019 (COVID-19) Outbreak in China: Summary of a Report of 72314 Cases From the Chinese Center for Disease Control and Prevention. JAMA. 2020.
4. Richardson S, Hirsch J S, Narasimhan M, et al. Presenting Characteristics, Comorbidities, and Outcomes Among 5700 Patients Hospitalized With COVID-19 in the New York City Area. JAMA. 2020.
5. Wu Q, Zhou L, Sun X, et al. Altered Lipid Metabolism in Recovered SARS Patients Twelve Years after Infection. Sci Rep. 2017; 7(1):9110.
6. Huang C, Wang Y, Li X, et al. Clinical features of patients infected with 2019 novel coronavirus in Wuhan, China. Lancet. 2020; 395(10223):497-506.
7. Pan H, Peto R, Henao-Restrepo A M, et al. Repurposed Antiviral Drugs for Covid-19—Interim WHO Solidarity Trial Results. N Engl J Med. 2020.
8. Pedersen S F, Ho Y C. SARS-CoV-2: a storm is raging. The Journal of clinical investigation. 2020.
9. Mehta P, McAuley D F, Brown M, et al. COVID-19: consider cytokine storm syndromes and immunosuppression. Lancet. 2020; 395(10229):1033-1034.
10. Ackermann M, Verleden S E, Kuehnel M, et al. Pulmonary Vascular Endothelialitis, Thrombosis, and Angiogenesis in Covid-19. N Engl J Med. 2020; 383(2):120-128.
11. Subramaniam S, Scharrer I. Procoagulant activity during viral infections. Front Biosci (Landmark Ed). 2018; 23:1060-1081.
12. Tang N, Li D, Wang X, Sun Z. Abnormal coagulation parameters are associated with poor prognosis in patients with novel coronavirus pneumonia. J Thromb Haemost. 2020; 18(4):844-847.
13. Levi M, Toh C H, Thachil J, Watson H G. Guidelines for the diagnosis and management of disseminated intravascular coagulation. British Committee for Standards in Haematology. Br J Haematol. 2009; 145(1):24-33.
14. Oxley T J, Mocco J, Majidi S, et al. Large-Vessel Stroke as a Presenting Feature of Covid-19 in the Young. N Engl J Med. 2020.
15. Li W, Yue H. Thymidine phosphorylase: A potential new target for treating cardiovascular disease. Trends Cardiovasc Med. 2018; 28(3):157-171.
16. Hamed E A, Zakary M M, Abdelal R M, Abdel Moneim E M. Vasculopathy in type 2 diabetes mellitus: role of specific angiogenic modulators. J Physiol Biochem. 2011; 67(3):339-349.
17. Miyazono K, Okabe T, Urabe A, Takaku F, Heldin C H. Purification and properties of an endothelial cell growth factor from human platelets. The Journal of biological chemistry. 1987; 262(9):4098-4103.
18. Desgranges C, Razaka G, Rabaud M, Bricaud H. Catabolism of thymidine in human blood platelets: purification and properties of thymidine phosphorylase. Biochimica et biophysica acta. 1981; 654(2):211-218.
19. Fox S B, Moghaddam A, Westwood M, et al. Platelet-derived endothelial cell growth factor/thymidine phosphorylase expression in normal tissues: an immunohistochemical study. J Pathol. 1995; 176(2):183-190.
20. van Kuilenburg A B, Zoetekouw L. Determination of thymidine phosphorylase activity in human blood cells and fibroblasts by a nonradiochemical assay using reversed-phase high-performance liquid chromatography. Nucleosides, nucleotides & nucleic acids. 2006; 25(9-11): 1261-1264.
21. Oh T, El Kouni M H. Distinct substrate specificity and physicochemical characterization of native human hepatic thymidine phosphorylase. PloS one. 2018; 13(8):e0202826.
22. Mori S, Takao S, Ikeda R, et al. Thymidine phosphorylase suppresses Fas-induced apoptotic signal transduction independent of its enzymatic activity. Biochemical and biophysical research communications. 2002; 295(2):300-305.
23. Li W, Chiba Y, Kimura T, et al. Transmyocardial laser revascularization induced angiogenesis correlated with the expression of matrix metalloproteinases and platelet-derived endothelial cell growth factor. Eur J Cardiothorac Surg. 2001; 19(2):156-163.
24. Li W, Tanaka K, Morioka K, et al. Long-term effect of gene therapy for chronic ischemic myocardium using platelet-derived endothelial cell growth factor in dogs. J Gene Med. 2008; 10(4):412-420.
25. Yamada N, Li W, Ihaya A, et al. Platelet-derived endothelial cell growth factor gene therapy for limb ischemia. J Vasc Surg. 2006; 44(6):1322-1328.
26. Koukourakis M I, Giatromanolaki A, O'Byrne K J, et al. Platelet-derived endothelial cell growth factor expression correlates with tumour angiogenesis and prognosis in non-small-cell lung cancer. British journal of cancer. 1997; 75(4):477-481.
27. Akiyama S, Furukawa T, Sumizawa T, et al. The role of thymidine phosphorylase, an angiogenic enzyme, in tumor progression. Cancer Sci. 2004; 95(11):851-857.
28. Nakajima Y, Madhyastha R, Maruyama M. 2-Deoxy-D-ribose, a downstream mediator of thymidine phosphorylase, regulates tumor angiogenesis and progression. Anticancer Agents Med Chem. 2009; 9(2):239-245.
29. Li W, Gigante A, Perez-Perez M J, et al. Thymidine phosphorylase participates in platelet signaling and promotes thrombosis. Circulation research. 2014; 115(12):997-1006.
30. Belcher A, Zulfiker A H M, Li O Q, Yue H, Gupta A S, Li W. Targeting Thymidine Phosphorylase With Tipiracil Hydrochloride Attenuates Thrombosis Without Increasing Risk of Bleeding in Mice. Arteriosclerosis, thrombosis, and vascular biology.0(0):ATVBAHA.120.315109.
31. Filbin M R, Mehta A, Schneider A M, et al. Longitudinal proteomic analysis of severe COVID-19 reveals survival-associated signatures, tissue-specific cell death, and cell-cell interactions. Cell Reports Medicine. 2021; 2(5):100287.
32. Filbin M R, Mehta A, Schneider A M, et al. Plasma proteomics reveals tissue-specific cell death and mediators of cell-cell interactions in severe COVID-19 patients. bioRxiv. 2020:2020.2011.2002.365536.
33. Russell C D, Valanciute A, Gachanja N N, et al. Tissue Proteomic Analysis Identifies Mechanisms and Stages of Immunopathology in Fatal COVID-19. Am J Respir Cell Mol Biol. 2021.
34. Kish T, Uppal P. Trifluridine/Tipiracil (Lonsurf) for the Treatment of Metastatic Colorectal Cancer. P T. 2016; 41(5):314-325.
35. Long W, Yang J, Li Z, et al. Abnormal Fibrinogen Level as a Prognostic Indicator in Coronavirus Disease Patients: A Retrospective Cohort Study. Frontiers in Medicine. 2021; 8(892).
36. Zhang Y, Xiao M, Zhang S, et al. Coagulopathy and Antiphospholipid Antibodies in Patients with Covid-19. N Engl J Med. 2020; 382(17):e38.
37. Chen L, Li X, Chen M, Feng Y, Xiong C. The ACE2 expression in human heart indicates new potential mechanism of heart injury among patients infected with SARS-CoV-2. Cardiovasc Res. 2020; 116(6): 1097-1100.
38. Barnes B J, Adrover J M, Baxter-Stoltzfus A, et al. Targeting potential drivers of COVID-19: Neutrophil extracellular traps. The Journal of experimental medicine. 2020; 217(6).
39. Zuo Y, Yalavarthi S, Shi H, et al. Neutrophil extracellular traps in COVID-19. JCI Insight. 2020.
40. Liu P P, Blet A, Smyth D, Li H. The Science Underlying COVID-19: Implications for the Cardiovascular System. Circulation. 2020.
41. Al-Samkari H, Karp Leaf R S, Dzik W H, et al. COVID-19 and coagulation: bleeding and thrombotic manifestations of SARS-CoV-2 infection. Blood. 2020; 136(4):489-500.
42. Lodigiani C, Iapichino G, Carenzo L, et al. Venous and arterial thromboembolic complications in COVID-19 patients admitted to an academic hospital in Milan, Italy. Thrombosis research. 2020; 191:9-14.
43. L AB, Pacioni C, Ponton S, Francavilla S, Cuzzoli A. Arterial Mesenteric Thrombosis as a Complication of SARS-CoV-2 Infection. Eur J Case Rep Intern Med. 2020; 7(5):001690.
44. Ranucci M, Ballotta A, Di Dedda U, et al. The procoagulant pattern of patients with COVID-19 acute respiratory distress syndrome. J Thromb Haemost. 2020.
45. Watson R A, Johnson D M, Dharia R N, Merli G J, Doherty J U. Anti-coagulant and anti-platelet therapy in the COVID-19 patient: a best practices quality initiative across a large health system. Hosp Pract (1995). 2020; 48(4):169-179.
46. Manne B K, Denorme F, Middleton E A, et al. Platelet gene expression and function in patients with COVID-19. Blood. 2020; 136(11):1317-1329.
47. Zaid Y, Puhm F, Allaeys I, et al. Platelets Can Associate With SARS-CoV-2 RNA and Are Hyperactivated in COVID-19. Circulation research. 2020; 127(11):1404-1418.
48. Hoffmann M, Kleine-Weber H, Schroeder S, et al. SARS-CoV-2 Cell Entry Depends on ACE2 and TMPRSS2 and Is Blocked by a Clinically Proven Protease Inhibitor. Cell. 2020; 181(2):271-280 e278.
49. Ulrich H, Pillat M M. CD147 as a Target for COVID-19 Treatment: Suggested Effects of Azithromycin and Stem Cell Engagement. Stem Cell Rev Rep. 2020.
50. Yan R, Zhang Y, Li Y, Xia L, Guo Y, Zhou Q. Structural basis for the recognition of SARS-CoV-2 by full-length human ACE2. Science. 2020; 367(6485):1444-1448.
51. Zhang S, Liu Y, Wang X, et al. SARS-CoV-2 binds platelet ACE2 to enhance thrombosis in COVID-19. J Hematol Oncol. 2020; 13(1):120.
52. Lan J, Ge J, Yu J, et al. Structure of the SARS-CoV-2 spike receptor-binding domain bound to the ACE2 receptor. Nature. 2020.
53. Wrapp D, Wang N, Corbett K S, et al. Cryo-EM structure of the 2019-nCoV spike in the prefusion conformation. Science. 2020; 367(6483):1260-1263.
54. Li F. Structure, Function, and Evolution of Coronavirus Spike Proteins. Annu Rev Virol. 2016; 3(1):237-261.
55. Abbattista M, Martinelli I, Peyvandi F. Comparison of adverse drug reactions among four COVID-19 vaccines in Europe using the EudraVigilance database: Thrombosis at unusual sites. J Thromb Haemost. 2021; 19(10):2554-2558.
56. Cari L, Alhosseini M N, Fiore P, et al. Cardiovascular, neurological, and pulmonary events following vaccination with the BNT162b2, ChAdOx1 nCoV-19, and Ad26.COV2.S vaccines: An analysis of European data. J Autoimmun. 2021; 125:102742.
57. Schultz N H, Søvoll I H, Michelsen A E, et al. Thrombosis and Thrombocytopenia after ChAdOx1 nCoV-19 Vaccination. New England Journal of Medicine. 2021; 384(22):2124-2130.
58. Greinacher A, Thiele T, Warkentin T E, Weisser K, Kyrle P A, Eichinger S. Thrombotic Thrombocytopenia after ChAdOx1 nCov-19 Vaccination. New England Journal of Medicine. 2021; 384(22):2092-2101.
59. Muir K-L, Kallam A, Koepsell S A, Gundabolu K. Thrombotic Thrombocytopenia after Ad26.COV2.S Vaccination. New England Journal of Medicine. 2021; 384(20):1964-1965.
60. Thrombosis With Thrombocytopenia After the Messenger RNA-1273 Vaccine. Annals of Internal Medicine.0(0):null.
61. Su P-H, Yu Y-C, Chen W-H, et al. Case Report: Vaccine-Induced Immune Thrombotic Thrombocytopenia in a Pancreatic Cancer Patient After Vaccination With Messenger RNA-1273. Frontiers in Medicine. 2021; 8 (1998).
62. Carli G, Nichele I, Ruggeri M, Barra S, Tosetto A. Deep vein thrombosis (DVT) occurring shortly after the second dose of mRNA SARS-CoV-2 vaccine. Intern Emerg Med. 2021; 16(3):803-804.
63. Fraga-Silva R A, Sorg B S, Wankhede M, et al. ACE2 activation promotes antithrombotic activity. Mol Med. 2010; 16(5-6):210-215.
64. Jha P K, Vijay A, Halu A, Uchida S, Aikawa M. Gene Expression Profiling Reveals the Shared and Distinct Transcriptional Signatures in Human Lung Epithelial Cells Infected With SARS-CoV-2, MERS-CoV, or SARS-CoV: Potential Implications in Cardiovascular Complications of COVID-19. Front Cardiovasc Med. 2020; 7:623012.
65. Li W, Yue H. Thymidine Phosphorylase Is Increased in COVID-19 Patients in an Acuity-Dependent Manner. Front Med (Lausanne). 2021; 8:653773.
66. Shaath H, Vishnubalaji R, Elkord E, Alajez N M. Single-Cell Transcriptome Analysis Highlights a Role for Neutrophils and Inflammatory Macrophages in the Pathogenesis of Severe COVID-19. Cells. 2020; 9(11).
67. Blanco-Melo D, Nilsson-Payant B E, Liu W-C, et al. SARS-CoV-2 launches a unique transcriptional signature from in vitro, ex vivo, and in vivo systems. bioRxiv. 2020:2020.2003.2024.004655.
68. Gerlach R L, Camp J V, Chu Y K, Jonsson C B. Early host responses of seasonal and pandemic influenza A viruses in primary well-differentiated human lung epithelial cells. PloS one. 2013; 8(11):e78912.
69. Filbin M R, Mehta A, Schneider A M, et al. Longitudinal proteomic analysis of severe COVID-19 reveals survival-associated signatures, tissue-specific cell death, and cell-cell interactions. Cell Rep Med. 2021; 2(5):100287.
70. Zou K H, O'Malley A J, Mauri L. Receiver-Operating Characteristic Analysis for Evaluating Diagnostic Tests and Predictive Models. Circulation. 2007; 115(5):654-657.
71. Connors J M, Levy J H. COVID-19 and its implications for thrombosis and anticoagulation. Blood. 2020.
72. Bao L, Deng W, Huang B, et al. The pathogenicity of SARS-CoV-2 in hACE2 transgenic mice. Nature. 2020; 583(7818):830-833.
73. Winkler E S, Bailey A L, Kafai N M, et al. SARS-CoV-2 infection of human ACE2-transgenic mice causes severe lung inflammation and impaired function. Nature Immunology. 2020; 21(11): 1327-1335.
74. Arce V M, Costoya J A. SARS-CoV-2 infection in K18-ACE2 transgenic mice replicates human pulmonary disease in COVID-19. Cell Mol Immunol. 2021; 18(3):513-514.
75. Subramaniam S, Hekman R M, Jayaraman A, et al. Platelet proteome analysis reveals an early hyperactive phenotype in SARS-CoV-2-infected humanized ACE2 mice. bioRxiv. 2021:2021.2008.2019.457020.
76. Li W, McIntyre T M, Silverstein R L. Ferric chloride-induced murine carotid arterial injury: A model of redox pathology. Redox Biol. 2013; 1:50-55.
77. Li W, Nieman M, Sen Gupta A. Ferric Chloride-induced Murine Thrombosis Models. J Vis Exp. 2016(115).
78. Lopez L C, Akman H O, Garcia-Cazorla A, et al. Unbalanced deoxynucleotide pools cause mitochondrial DNA instability in thymidine phosphorylase-deficient mice. Human molecular genetics. 2009; 18(4):714-722.
79. Issa N, Lacassin F, Camou F. First case of persistent pancytopenia associated with SARS-CoV-2 bone marrow infiltration in an immunocompromised patient. Annals of oncology: official journal of the European Society for Medical Oncology/ESMO. 2020; 31(10):1418-1419.
80. Deinhardt-Emmer S, Wittschieber D, Sanft J, et al. Early postmortem mapping of SARS-CoV-2 RNA in patients with COVID-19 and correlation to tissue damage. bioRxiv. 2020:2020.2007.2001.182550.
81. Kurre P, Anandakumar P, Kiem H P. Rapid 1-hour transduction of whole bone marrow leads to long-term repopulation of murine recipients with lentivirus-modified hematopoietic stem cells. Gene therapy. 2006; 13(4):369-373.
82. Lefrancais E, Ortiz-Munoz G, Caudrillier A, et al. The lung is a site of platelet biogenesis and a reservoir for haematopoietic progenitors. Nature. 2017; 544(7648):105-109.
83. Gupta N, Li W, Willard B, Silverstein R L, McIntyre T M. Proteasome proteolysis supports stimulated platelet function and thrombosis. Arteriosclerosis, thrombosis, and vascular biology. 2014; 34(1):160-168.
84. Li W, Nieman, M., Sen Gupta, A. Ferric Chloride-induced Murine Thrombosis Models. J Vis Exp. 2016; 115:e54479.
85. Chen K, Febbraio M, Li W, Silverstein R L. A specific CD36-dependent signaling pathway is required for platelet activation by oxidized low-density lipoprotein. Circulation research. 2008; 102(12):1512-1519.
86. Chen K, Li W, Major J, Rahaman S O, Febbraio M, Silverstein R L. Vav guanine nucleotide exchange factors link hyperlipidemia and a prothrombotic state. Blood. 2011; 117(21):5744-5750.
87. Yang M, Li W, Harberg C, et al. Cysteine sulfenylation by CD36 signaling promotes arterial thrombosis in dyslipidemia. Blood Adv. 2020; 4(18):4494-4507.
88. Siddamreddy S, Thotakura R, Dandu V, Kanuru S, Meegada S. Corona Virus Disease 2019 (COVID-19) Presenting as Acute ST Elevation Myocardial Infarction. Cureus. 2020; 12(4):e7782.
89. Wilhelmus K R. Antiviral treatment and other therapeutic interventions for herpes simplex virus epithelial keratitis. Cochrane Database Syst Rev. 2015; 1:CD002898.
90. Bijnsdorp I V, Kruyt F A, Fukushima M, Peters G J. Trifluorothymidine induces cell death independently of p53. Nucleosides, nucleotides & nucleic acids. 2008; 27(6):699-703.
91. Lu R, Zhao X, Li J, et al. Genomic characterisation and epidemiology of 2019 novel coronavirus: implications for virus origins and receptor binding. Lancet. 2020; 395(10224):565-574.
92. Kim Y, Wower J, Maltseva N, et al. Tipiracil binds to uridine site and inhibits Nsp15 endoribonuclease NendoU from SARS-CoV-2. Commun Biol. 2021; 4(1):193.
93. Shen S, Zhang J, Fang Y, et al. SARS-CoV-2 interacts with platelets and megakaryocytes via ACE2-independent mechanism. Journal of Hematology & Oncology. 2021; 14(1):72.
94. Amraei R, Yin W, Napoleon M A, et al. CD209L/L-SIGN and CD209/DC-SIGN Act as Receptors for SARS-CoV-2. ACS Central Science. 2021; 7(7):1156-1165.
95. Jeffers S A, Tusell S M, Gillim-Ross L, et al. CD209L (L-SIGN) is a receptor for severe acute respiratory syndrome coronavirus. Proceedings of the National Academy of Sciences of the United States of America. 2004; 101(44):15748-15753.
96. Ichimura T, Mori Y, Aschauer P, et al. KIM-1/TIM-1 is a Receptor for SARS-CoV-2 in Lung and Kidney. medRxiv. 2020:2020.2009.2016.20190694.
97. Wang S, Qiu Z, Hou Y, et al. AXL is a candidate receptor for SARS-CoV-2 that promotes infection of pulmonary and bronchial epithelial cells. Cell Research. 2021; 31(2):126-140.
98. Choudhury A, Mukherjee S. In silico studies on the comparative characterization of the interactions of SARS-CoV-2 spike glycoprotein with ACE-2 receptor homologs and human TLRs. J Med Virol. 2020.
99. Heinzmann D, Noethel M, Ungern-Sternberg S V, et al. CD147 is a Novel Interaction Partner of Integrin alphaMbeta2 Mediating Leukocyte and Platelet Adhesion. Biomolecules. 2020; 10(4).
100. Wang K, Chen W, Zhang Z, et al. CD147-spike protein is a novel route for SARS-CoV-2 infection to host cells. Signal Transduction and Targeted Therapy. 2020; 5(1):283.
101. Schuurs Z P, Hammond E, Elli S, et al. Evidence of a putative glycosaminoglycan binding site on the glycosylated SARS-CoV-2 spike protein N-terminal domain. Computational and Structural Biotechnology Journal. 2021; 19:2806-2818.
102. Shilts J, Crozier T W M, Greenwood E J D, Lehner P J, Wright G J. No evidence for basigin/CD147 as a direct SARS-CoV-2 spike binding receptor. Scientific Reports. 2021; 11(1):413.
103. Gordon D E, Jang G M, Bouhaddou M, et al. A SARS-CoV-2 protein interaction map reveals targets for drug repurposing. Nature. 2020; 583(7816):459-468.
104. Burkard C, Verheije M H, Haagmans B L, et al. ATP1A1-Mediated Src Signaling Inhibits Coronavirus Entry into Host Cells. Journal of Virology. 2015; 89(8):4434-4448.
105. Li X, Ma X. Acute respiratory failure in COVID-19: is it “typical” ARDS? Crit Care. 2020; 24(1):198.
106. Wichmann D, Sperhake J P, Lutgehetmann M, et al. Autopsy Findings and Venous Thromboembolism in Patients With COVID-19: A Prospective Cohort Study. Ann Intern Med. 2020.
107. Guillot L, Nathan N, Tabary O, et al. Alveolar epithelial cells: master regulators of lung homeostasis. The international journal of biochemistry & cell biology. 2013; 45(11):2568-2573.
108. Ziegler C G K, Allon S J, Nyquist S K, et al. SARS-CoV-2 Receptor ACE2 Is an Interferon-Stimulated Gene in Human Airway Epithelial Cells and Is Detected in Specific Cell Subsets across Tissues. Cell. 2020; 181(5):1016-1035.e1019.
109. Lukassen S, Chua R L, Trefzer T, et al. SARS-CoV-2 receptor ACE2 and TMPRSS2 are primarily expressed in bronchial transient secretory cells. The EMBO Journal. 2020; 39(10):e105114.
110. Tombolini A, Scendoni R. SARS-CoV-2-related deaths in routine forensic autopsy practice: histopathological patterns. Int J Legal Med. 2020; 134(6):2205-2208.
111. Carsana L, Sonzogni A, Nasr A, et al. Pulmonary post-mortem findings in a series of COVID-19 cases from northern Italy: a two-centre descriptive study. Lancet Infect Dis. 2020; 20(10):1135-1140.
112. Waguri Y, Otsuka T, Sugimura I, et al. Gliostatin/platelet-derived endothelial cell growth factor as a clinical marker of rheumatoid arthritis and its regulation in fibroblast-like synoviocytes. Br J Rheumatol. 1997; 36(3):315-321.
113. Toyoda Y, Tabata S, Kishi J, et al. Thymidine phosphorylase regulates the expression of CXCL10 in rheumatoid arthritis fibroblast-like synoviocytes. Arthritis Rheumatol. 2014; 66(3):560-568.
114. Rahmati M, Petitbarat M, Dubanchet S, Bensussan A, Chaouat G, Ledee N. Granulocyte-Colony Stimulating Factor related pathways tested on an endometrial ex-vivo model. PloS one. 2014; 9(9):e102286.
115. Nakajima Y, Gotanda T, Uchimiya H, et al. Inhibition of metastasis of tumor cells overexpressing thymidine phosphorylase by 2-deoxy-L-ribose. Cancer research. 2004; 64(5):1794-1801.
116. Tay M Z, Poh C M, Renia L, MacAry P A, Ng L F P. The trinity of COVID-19: immunity, inflammation and intervention. Nature Reviews Immunology. 2020.
117. Xiong Y, Liu Y, Cao L, et al. Transcriptomic characteristics of bronchoalveolar lavage fluid and peripheral blood mononuclear cells in COVID-19 patients. Emerg Microbes Infect. 2020; 9(1):761-770.
118. Guo L, Rondina M T. The Era of Thromboinflammation: Platelets Are Dynamic Sensors and Effector Cells During Infectious Diseases. Front Immunol. 2019; 10:2204.
119. van de Veerdonk F L, Netea M G, van Deuren M, et al. Kallikrein-kinin blockade in patients with COVID-19 to prevent acute respiratory distress syndrome. Elife. 2020; 9.
120. Harrison A G, Lin T, Wang P. Mechanisms of SARS-CoV-2 Transmission and Pathogenesis. Trends Immunol. 2020; 41(12):1100-1115.
121. El-Hashim A Z, Renno W M, Raghupathy R, Abduo H T, Akhtar S, Benter I F. Angiotensin-(1-7) inhibits allergic inflammation, via the MAS1 receptor, through suppression of ERK1/2- and NF-kappaB-dependent pathways. British journal of pharmacology. 2012; 166(6):1964-1976.
122. Rodrigues-Machado M G, Magalhaes G S, Cardoso J A, et al. AVE 0991, a non-peptide mimic of angiotensin-(1-7) effects, attenuates pulmonary remodelling in a model of chronic asthma. British journal of pharmacology. 2013; 170(4):835-846.
123. Jia H P, Look D C, Shi L, et al. ACE2 receptor expression and severe acute respiratory syndrome coronavirus infection depend on differentiation of human airway epithelia. J Virol. 2005; 79(23): 14614-14621.
124. Bayati A, Kumar R, Francis V, McPherson P S. SARS-CoV-2 infects cells after viral entry via clathrin-mediated endocytosis. Journal of Biological Chemistry. 2021; 296:100306.
125. Inoue Y, Tanaka N, Tanaka Y, et al. Clathrin-dependent entry of severe acute respiratory syndrome coronavirus into target cells expressing ACE2 with the cytoplasmic tail deleted. J Virol. 2007; 81(16):8722-8729.
126. de Wit E, van Doremalen N, Falzarano D, Munster V J. SARS and MERS: recent insights into emerging coronaviruses. Nature Reviews Microbiology. 2016; 14(8):523-534.
127. Li G, Fan Y, Lai Y, et al. Coronavirus infections and immune responses. Journal of Medical Virology. 2020; 92(4):424-432.
128. Gao W, Xiong Y, Li Q, Yang H. Inhibition of Toll-Like Receptor Signaling as a Promising Therapy for Inflammatory Diseases: A Journey from Molecular to Nano Therapeutics. Front Physiol. 2017; 8:508.
129. Kim S, Joe Y, Surh Y J, Chung H T. Differential Regulation of Toll-Like Receptor-Mediated Cytokine Production by Unfolded Protein Response. Oxid Med Cell Longev. 2018; 2018:9827312.
130. Li W, Tanaka K, Morioka K, et al. Thymidine phosphorylase gene transfer inhibits vascular smooth muscle cell proliferation by upregulating heme oxygenase-1 and p27KIP1. Arteriosclerosis, thrombosis, and vascular biology. 2005; 25(7):1370-1375.
131. Yue H, Tanaka K, Furukawa T, Karnik S S, Li W. Thymidine phosphorylase inhibits vascular smooth muscle cell proliferation via upregulation of STAT3. Biochimica et biophysica acta. 2012; 1823(8):1316-1323.
132. Christian F, Smith E L, Carmody R J. The Regulation of NF-kappaB Subunits by Phosphorylation. Cells. 2016; 5(1).
133. Chapouly C, Tadesse Argaw A, Horng S, et al. Astrocytic TYMP and VEGFA drive blood—brain barrier opening in inflammatory central nervous system lesions. Brain. 2015; 138(6):1548-1567.
134. Economides A N, Frendewey D, Yang P, et al. Conditionals by inversion provide a universal method for the generation of conditional alleles. Proceedings of the National Academy of Sciences of the United States of America. 2013; 110(34):E3179-3188.
135. Chapman H A, Li X, Alexander J P, et al. Integrin alpha6beta4 identifies an adult distal lung epithelial population with regenerative potential in mice. The Journal of clinical investigation. 2011; 121(7):2855-2862.
136. Gillich A, Zhang F, Farmer C G, et al. Capillary cell-type specialization in the alveolus. Nature. 2020; 586(7831):785-789.
137. Buckley S M, Howe S J, Sheard V, et al. Lentiviral transduction of the murine lung provides efficient pseudotype and developmental stage-dependent cell-specific transgene expression. Gene therapy. 2008; 15(16):1167-1175.
138. Helms M N, Torres-Gonzalez E, Goodson P, Rojas M. Direct tracheal instillation of solutes into mouse lung. J Vis Exp. 2010(42).
139. Valdivia-Mazeyra M F, Salas C, Nieves-Alonso J M, et al. Increased number of pulmonary megakaryocytes in COVID-19 patients with diffuse alveolar damage: an autopsy study with clinical correlation and review of the literature. Virchows Archiv: an international journal of pathology. 2020.
140. Edler C, Schroder A S, Aepfelbacher M, et al. Dying with SARS-CoV-2 infection-an autopsy study of the first consecutive 80 cases in Hamburg, Germany. Int J Legal Med. 2020; 134(4):1275-1284.
141. Oladunni F S, Park J G, Pino P A, et al. Lethality of SARS-CoV-2 infection in K18 human angiotensin-converting enzyme 2 transgenic mice. Nat Commun. 2020; 11(1):6122.
142. Zheng J, Wong L R, Li K, et al. COVID-19 treatments and pathogenesis including anosmia in K18-hACE2 mice. Nature. 2021; 589(7843):603-607.
143. Srikanthan S, Li W, Silverstein R L, McIntyre T M. Exosome poly-ubiquitin inhibits platelet activation, downregulates CD36 and inhibits pro-atherothombotic cellular functions. J Thromb Haemost. 2014; 12(11): 1906-1917.
144. Belcher A, Zulfiker A H M, Li O Q, Yue H, Gupta A S, Li W. Targeting Thymidine Phosphorylase With Tipiracil Hydrochloride Attenuates Thrombosis Without Increasing Risk of Bleeding in Mice. Arteriosclerosis, thrombosis, and vascular biology. 2021; 41(2):668-682.
145. Fang Y, Gao F, Liu Z. Angiotensin-converting enzyme 2 attenuates inflammatory response and oxidative stress in hyperoxic lung injury by regulating NF-kappaB and Nrf2 pathways. QJM. 2019; 112(12):914-924.
146. Guan W J, Ni Z Y, Hu Y, et al. Clinical Characteristics of Coronavirus Disease 2019 in China. N Engl J Med. 2020; 382(18):1708-1720.
147. Lauer S A, Grantz K H, Bi Q, et al. The Incubation Period of Coronavirus Disease 2019 (COVID-19) From Publicly Reported Confirmed Cases: Estimation and Application. Ann Intern Med. 2020; 172(9):577-582.
148. Ghosh A, Li W, Febbraio M, et al. Platelet CD36 mediates interactions with endothelial cell-derived microparticles and contributes to thrombosis in mice. The Journal of clinical investigation. 2008; 118(5):1934-1943.
149. Klenotic P A, Page R C, Li W, Amick J, Misra S, Silverstein R L. Molecular basis of antiangiogenic thrombospondin-1 type 1 repeat domain interactions with CD36. Arteriosclerosis, thrombosis, and vascular biology. 2013; 33(7):1655-1662.
150. Li W, Febbraio M, Reddy S P, Yu D Y, Yamamoto M, Silverstein R L. CD36 participates in a signaling pathway that regulates ROS formation in murine VSMCs. The Journal of clinical investigation. 2010; 120(11):3996-4006.
151. Yue H, Li W, Desnoyer R, Karnik S S. Role of nuclear unphosphorylated STAT3 in angiotensin II type 1 receptor-induced cardiac hypertrophy. Cardiovasc Res. 2010; 85(1):90-99.
152. Li W, Tanaka K, Ihaya A, et al. Gene therapy for chronic myocardial ischemia using platelet-derived endothelial cell growth factor in dogs. Am J Physiol Heart Circ Physiol. 2005; 288(1):H408-415.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method for diagnosis or prognosis of COVID-19 acuity in a subject having COVID-19, comprising:
(a) providing a biological sample from the subject;
(b) determining an expression level or activity of thymidine phosphorylase in the sample; and
(c) comparing the expression level or activity of thymidine phosphorylase in the sample, if present, to a control expression level or activity of thymidine phosphorylase, wherein the subject is diagnosed as having or at an increased risk of COVID-19 acuity if there is a measurable difference in the expression level or activity of the thymidine phosphorylase in the sample as compared to the control level.

2. The method of claim 1, wherein the COVID-19 acuity is characterized by thrombosis, inflammation, and/or organ damage in the subject.

3. The method of claim 1, wherein the biological sample comprises blood, plasma, or serum.

4. The method of claim 1, wherein the subject is human.

5. The method of claim 1, wherein determining the expression level or activity in the sample of the thymidine phosphorylase comprises determining the expression level or activity in the sample of the thymidine phosphorylase using mass spectrometry (MS) analysis, immunoassay analysis, or both.

6. The method of claim 1, further comprising selecting a treatment or modifying a treatment for the COVID-19 based on the determined expression level or activity of the thymidine phosphorylase.

7. The method of claim 1, further comprising determining an expression level or activity of C-reactive protein (CRP) and/or lactate dehydrogenase (LDH) in the sample.

8. The method of claim 1, further comprising administering to the subject a therapeutic agent capable of affecting the expression level or activity of thymidine phosphorylase in the subject.

9. The method of claim 8, wherein the therapeutic agent is tipiracil.

10. A method for determining whether to initiate or continue prophylaxis or treatment of COVID-19 in a subject, comprising:
(a) providing a series of biological samples over a time period from the subject;
(b) analyzing the series of biological samples to determine an expression level or activity in each of the biological samples of thymidine phosphorylase; and
(c) comparing any measurable change in the expression level or activity of thymidine phosphorylase in each of the biological samples to thereby determine whether to initiate or continue the prophylaxis or therapy of the COVID-19.

11. The method of claim 10, further comprising determining an expression level or activity of C-reactive protein (CRP) and/or lactate dehydrogenase (LDH) in the sample.

12. The method of claim 11, further comprising determining whether to initiate or continue prophylaxis or therapy of the COVID-19 based on the expression level or activity of CRP or LDH in the sample.

13. The method of claim 10, further comprising administering to the subject a therapeutic agent capable of affecting the expression level or activity of thymidine phosphorylase in the subject.

14. The method of claim 13, wherein the therapeutic agent is tipiracil.

15. A method for treating COVID-19 in a subject, comprising:
identifying a subject as having an increased expression level and/or activity of thymidine phosphorylase in a biological sample obtained from the subject; and
administering an effective amount of a therapeutic agent that reduces the expression level or activity of thymidine phosphorylase in the subject.

16. The method of claim 15, wherein reducing the expression level or activity of thymidine phosphorylase comprises administering to the subject an effective amount of a thymidine phosphorylase inhibitor.

17. The method of claim 16, wherein the thymidine phosphorylase inhibitor is tipiracil.

18. The method of claim 15, wherein administering the effective amount of the therapeutic agent reduces an amount of inflammation, D-dimer formation, organ damage, and/or thrombosis in the subject.

19. An assay for assessing COVID-19 acuity in a subject, comprising:
applying an agent capable of affecting detection of an expression level or activity of thymidine phosphorylase in a biological sample obtained from a subject; and
determining the expression level or activity of thymidine phosphorylase in the biological sample.

20. A method for screening for a compound useful for treating COVID-19, comprising:
contacting a cell with an effective amount of a test compound; and
detecting an expression level or activity level of thymidine phosphorylase in the cell in the presence of the test compound.
